SEQUENCE LISTING (25 sequences)

SEQ ID NO: 1 - protein, 665 aa, Streptococcus mitis
MNQEKRLHRFVKKCGLGVCSAVVAAFLLNAQGVALATEQGNRPVETENIARGKQASQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVELKTKNTPLSLAEVEVFRSATTQVGQDRTAPVVDQTSALKDYLFGLTYNPLDILTRKGETLENRYNTSAKEQNGEFVVVEKIKKTLSTGTADVSINGNQNVFLGGLYKANQNLLENQPELISLARAKGTVSVDLPGMIHSENKIEANPTTSGMQEAMNTLVEKWTKNYSSSHSVPARVQYESTTAYSMNQLKAKFGADFEKAGAPLKIDFEAVQKGEKQIEVVNFKQIYYTATFDAPTNPAAVFDKSVTPEDLKQRGVDSQTPPVYVSNVSYGRQIYVKFESTSKSTELKAAINAVIKGATIAPNSEWSRLLKNTSVTAVIVGGNASGAAKVVTGTVENLKELIREGANFSAQSPAVPISYKTAFLKDNAQATLQNSTDYIETKVTSYKNGFLKLHHKGAYIARYYIYWDEITYDEQGNPEIRSRQWEDNGKNRTSGFQTEIQFRGNVRNLRIKVQEKTGLVWEPWRTVYNRTDLPLVQQRTITHWGTTLNPKVDEKIVNE

SEQ ID NO: 2 - DNA, 1998 bp, Streptococcus mitis
atgaatcaagaaaaacgtttgcatcgctttgtcaaaaagtgtggactcggtgtgtgtagtgctgttgttgcggcctttttattgaacgctcagggagtagctttggctacagagcaagggaatcgtccagttgagaccaaaaacatcgctcgtggaaaacaagctagtcaaagttctactgcttatggaggagctgctacacgagcagtggacggtaatgttgacagtgactatgggcaccattctgtaacgcacacaaactttgaagacaatgcttggtggcaagttgatcttggaaaaacagagaatgttggaaaagttaaactctacaatcgtggagatgggaatgtagctaatcgtctttccaattttgatgttgttttgttaaatgaagcaaaacaagaagttgctcgtcaacactttgatagtttgaatgggaaggcagaacttgaagttttcttcaccgccaaagacgctcgttatgtaaaagtggagttgaaaactaaaaataccccattgagcttggcagaagtggaagtattccgttcagcaacaactcaagttggacaggatagaactgcaccagtagtagatcaaacatcagcattgaaagactacctttttggcttaacttataatcctttggatattttaactcgcaagggagaaaccttagaaaatcgctacaacacaagtgctaaggaacaaaatggagagtttgtcgttgtagaaaaaatcaagaaaaccctctctacaggcacagcagatgtttccatcaatggaaatcaaaatgtcttcctaggtggcttgtataaggcaaaccaaaatctgctagaaaatcagccagaattgattagtcttgcccgtgcaaaggggacagtcagcgtcgatttacctggtatgattcactctgaaaacaaaattgaagcaaatcctacaactagtggtatgcaggaggcgatgaataccttggttgaaagatggacaaagaattactcatctagccattccgttcctgcccgtgttcagtatgaatcaactacagcctatagcatgaatcaattgaaagcaaaatttggtgcagactttgaaaaagcaggtgcaccgctcaagattgactttgaggctgtgcaaaagggtgaaaagcaaattgaagttgtaaactttaaacaaatctactatacagcgacatttgatgcaccgaccaatccagcggctgtatttgacaagagtgtgacacctgaagatttaaaacaaagaggcgttgattcacaaactccacctgtatatgtatcaaatgtttcttacggacgtcaaatctatgttaagtttgagtcaacaagtaagtctactgaattaaaagcagctattaatgcggttatcaaaggcgcaacaattgctccaaattctgaatggagccgtctattgaagaatacttctgtaacagcggtaattgtaggaggtaatgctagcggtgccgccaaagttgtcacaggaacagtcgaaaacttgaaggaactcatcagagaaggagctaactttagcgctcaaagtccagctgtgccaatctcatataagactgccttcctaaaagataatgcccaagcaactttacaaaatagtacagactatatcgaaacgaaggttacttcttacaaaaatggtttcttgaaacttcatcataagggtgcttatattgcgcgttactacatctattgggatgaaattacatatgatgaacaaggaaatccagaaatccgttcacgtcaatgggaagataacggaaaaaacagaacttcaggcttccaaacagagattcaatttagaggaaatgtccgtaatcttcgcatcaaggttcaagaaaagacgggtcttgtatgggaaccatggcgtacagtttacaaccgcacagacttaccactagtacaacagcgtacaattacacattggggaacaactctaaaccctaaagttgatgaaaaaattgtgaatgagtaa

SEQ ID NO: 3 - protein, 162 aa, Streptococcus mitis
TEQGNRPVETENIARGKQASQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVELKTKNTPLSLAEVEVFRSATTQVGQDRTAPVVD

SEQ ID NO: 4 - DNA, 486 bp, Streptococcus mitis
acagagcaagggaatcgtccagttgagaccgaaaacatcgctcgtggaaaacaagctagtcaaagttctactgcttatggaggagctgctgcacgagcagtggacggtaatgttgacagtgactatgggcaccattctgtaacgcacacaaactttgaagacaatgcttggtggcaagttgatcttggaaaaacagagaatgttggaaaagttaaactctacaatcgtggagatgggaatgtagctaatcgtctttccaattttgatgttgttttgttaaatgaagcaaaacaagaagttgctcgtcaacactttgatagtttgaatgggaaggcagaacttgaagttttcttcaccgccaaagccgctcgttatgtaaaagtggagttgaaaactaaaaataccccattgagcttggcagaagtggaagtattccgttcagcaacaactcaagttggacaggatagaactgcaccagtagtagat

SEQ ID NO: 5 - DNA, 39 bp, Streptococcus mitis
atgaatcaagaaaaacgtttgcatcgctttgtcaaaaag

SEQ ID NO: 6 - DNA, 39 bp, Streptococcus mitis
ttactcattcacaattttttcatcaactttagggtttag

SEQ ID NO: 7 - DNA, 30 bp, Streptococcus mitis
tcggatccgagcaagggaatcgtccagttg

SEQ ID NO: 8 - DNA, 42 bp, Streptococcus mitis
ctgaattcttactcattcacaattttttcatcaactttaggg

SEQ ID NO: 9 - protein, 532 aa, Streptococcus intermedius
MKTKQNIARKLSRVVLLSTLVLSSAAPISAAFAETPTKPKAAQTEKKPEKKPENSNSEAAKKALNDYIWGLQYDKLNILTHQGEKLKNHSSREAFHRPGEYVVIEKKKQSISNATSKLSVSSANDDRIFPGALLKADQSLLENLPTLIPVNRGKTTISVNLPGLKNGESNLTVENPSNSTVRTAVNNLVEKWIQKYSKTHAVPARMQYESISAQSMSQLQAKFGADFSKVGAPLNVDFSSVHKGEKQVFIANFRQVYYTASVDSPNSPSALFGSGITPTDLINRGVNSKTPPVYVSNVSYGRAMYVKFETTSKSTKVQAAIDAVVKGAKLKAGTEYENILKNTKITAVVLGGNPGEASKVITGNIDTLKDLIQKGSNFSAQSPAVPISYTTSFVKDNSIATIQNNTDYIETKVTSYKDGALTLNHDGAFVARFYVYWEELGHDADGYETIRSRSWSGNGYNRGAHYSTTLRFKGNVRNIRVKVLGATGLAWEPWRLIYSKNDLPLVPQRNISTWGTTLHPQFEDKVVKDNTD

SEQ ID NO: 10 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 11 - protein, 158 aa, Anguilla anguilla
VIPEGYTQENVAVRGKATQSAQLRGEHAANSEASNAIDGNRDSNFYHGSCTHSSGQANPWWRVDLLQVYTITSVTITNRGDCCGERISGAEINIGQHLASNGVNNPECSVIGSMATGETKTFHCPAPMIGRYVVTYLPTSESLHLCEVEVNVDKPAAA

SEQ ID NO: 12 - protein, 160 aa, Streptococcus pneumoniae
EEDIDKITEDKVVSTNKVATQSSTNYEGVAALAVDGNKDGDYGHHSVTHTKADSNAWWQVDLGEEFTVSKVDIYNRTDAEPQRLSNFDVIFLSSSGEEVFRRHFDKVADGLLSLKVPSVGAKLVKIELKSAAIPLSLAEVEVYGSKRTPKKLSNIALTKE

SEQ ID NO: 13 - protein, 11 aa, Streptococcus mitis
EKTGLVWEPWR

SEQ ID NO: 14 - protein, 11 aa, Streptococcus pneumoniae
ECTGLAWEWWR

SEQ ID NO: 15 - protein, 11 aa, Streptococcus intermedius
GATGLAWEPWR

SEQ ID NO: 16 - protein, 46 aa, Streptococcus mitis
HSVTHTNFEDNAWWQVDLGKTENVGKVKLYNRGDGNVANRLSNFDV

SEQ ID NO: 17 - protein, 56 aa, Streptococcus mitis
SDYGHHSVTHTNFEDNAWWQVDLGKTENVGKVKLYNRGDGNVANRLSNFDVVLLNE

SEQ ID NO: 18 - protein, 66 aa, Streptococcus mitis
DGNVDSDYGHHSVTHTNFEDNAWWQVDLGKTENVGKVKLYNRGDGNVANRLSNFDVVLLNEAKQEV

SEQ ID NO: 19 - protein, 81 aa, Streptococcus mitis
ATRAVDGNVDSDYGHHSVTHTNFEDNAWWQVDLGKTENVGKVKLYNRGDGNVANRLSNFDVVLLNEAKQEVARQHFDSLNG

SEQ ID NO: 20 - protein, 96 aa, Streptococcus mitis
AYGGAATRAVDGNVDSDYGHHSVTHTNFEDNAWWQVDLGKTENVGKVKLYNRGDGNVANRLSNFDVVLLNEAKQEVARQHFDSLNGKAELEVFFTA

SEQ ID NO: 21 - protein, 111 aa, Streptococcus mitis
SQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVEL

SEQ ID NO: 22 - protein, 126 aa, Streptococcus mitis
RGKQASQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVELKTKNTPLSLA

SEQ ID NO: 23 - protein, 141 aa, Streptococcus mitis
TENIARGKQASQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVELKTKNTPLSLAEVEVFRSATT

SEQ ID NO: 24 - protein, 151 aa, Streptococcus mitis
NRPVETENIARGKQASQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVELKTKNTPLSLAEVEVFRSATTQVGQD

SEQ ID NO: 25 - protein, 158 aa, Streptococcus mitis
QGNRPVETENIARGKQASQSSTAYGGAATRAVDGNVDSDYGHHSVTHTNFEDNAWWQVDLGKTENVGKVKLYNRGDGNVANRLSNFDVVLLNEAKQEVARQHFDSLNGKAELEVFFTAKDARYVKVELKTKNTPLSLAEVEVFRSATTQVGQDRTAPV

(19) United States
(12) Patent Application Publication
(10) Pub. No.: US 2011/0052494 A1
(43) Pub. Date: Mar. 3, 2011

(54) H-ANTIGEN BINDING POLYPEPTIDES AND METHODS OF USE

(76) Inventor: RODNEY K. TWETEN, EDMOND, OK (US)

(21) Appl. No.: 12/669,784

(22) Filed: Jan. 19, 2010

(30) Foreign Application Priority Data
Jul. 21, 2008 (US) PCT/US2008/070653

(51) Int. Cl. A61K 49/00; C07K 14/00; C07H 21/00; C12N 15/63; C12N 5/10; A61K 38/16; G01N 33/574; A61P 35/00

(52) U.S. Cl. 424/9.1; 530/350; 530/324; 536/23.1; 435/320.1; 435/325; 514/19.3; 435/7.23

(57) ABSTRACT

Polypeptides having high specific binding for H-antigen, particularly Lewis y and Lewis b antigens are described. The polypeptides are useful in making conjugates with diagnostic reporter molecules or therapeutic agents such as anticancer agents for binding to epithelial derived tumors or cancer cells which overexpress such antigens.

### CROSS-REFERENCE TO RELATED APPLICATIONS

The present application claims the benefit under 35 U.S.C. 119(e) of U.S. Provisional Application Ser. No. 60/961,135, filed Jul. 19, 2007 and U.S. patent application Ser. No. 12/102,696, filed Apr. 14, 2008. The present application also claims priority based on International Patent Application PCT/US2008/070653 filed on Jul. 21, 2008. The entirety of each of these applications is hereby expressly incorporated herein by reference.

### BACKGROUND

Abnormal cell proliferation is usually characterized by an increased rate of division and in some cases uncontrolled growth. One example of a proliferative cell disorder is a tumor and particularly a malignant tumor. Primary malignant tumors are especially problematic given their tendency to invade surrounding tissues and metastasize to distant organs in the body. To date, the most frequently used methods for treating neoplasias, especially solid tumor forms of neoplasia, include surgical procedures, radiation therapy, drug therapies, and combinations of the foregoing. These methods involve significant risk to the patient. Moreover, the probability of eliminating all malignant cells is small particularly if the zone of malignant growth is not well defined or if the primary tumor has metastasized by the time of surgery. Achieving therapeutic doses effective for treating the cancer is often limited by the toxic side effects of the anticancer agent on normal, healthy tissues. An ideal anticancer agent has tissue specificity, in particular for abnormal tissues, thereby reducing side-effects on normal, noncancerous cells.

It is thus a primary goal of medicine to be able to specifically direct therapeutic agents such as anticancer agents directly to the site of the tumor or malignant cells.

H-antigens are indirect gene products expressed as glycan units containing fucose in α1-2 linkage to galactose, residing on glycoproteins or glycolipids of erythrocyte membranes or on mucin glycoproteins in secretions. They are the fucosylated glycans that are the direct substrates for glycosyltransferases that give rise to the epitopes for the A, B and Lewis blood group antigens. Typically the only cells that carry H-antigen on their surfaces are erythrocytes (of the ABO and Lewis blood groups). Type O erythrocytes typically display the H-antigen, for example. Hence expression of other H-antigen-containing cells is limited and is often associated with abnormalities such as cancer. Two different, closely linked loci encode the fucosyltransferases that give rise to the Hh antigens; both loci encode closely homologous α1,2 fucosyltransferases (Fut1 and Fut2) that transfer fucose in an α1-2 linkage to a galactose and result in products whose structure is nearly identical (differing only by a single carbohydrate linkage that differentiates type 1 from type 2 glycans) and constitutes the epitope for the H-antigen. Fut1 is preferentially expressed in erythroid tissues and uses type 2 chains as substrates, whereas Fut2 is predominantly expressed in secretory epithelial cells and acts preferentially on type 1 glycans.

However, it is known that H-antigens are often upregulated in epithelial tumor types and changes in these glycans levels are often associated with a poor clinical prognosis. For example, Le^{y} is expressed on 60-90% of epithelial cancers, including cancers of the breast, pancreas, ovary, colon, gastric, and lung.

The identification of a ligand able to specifically bind with high affinity to the Lewis y and Lewis b glycans overexpressed on tumor cell surfaces would be a useful discovery in the search for treatments against such cancers.

### BRIEF DESCRIPTION OF THE DRAWINGS

FIG. 1. Primary structure of LLY. (a) The primary structure of LLY (SEQ ID NO:1) and its comparison with the primary structures of ILY (SEQ ID NO:9) and PLY (SEQ ID NO:10). Comparison of the primary structure of LLY^{Lec} with that of the (b) A. anguilla agglutinin (AAA) (SEQ ID NO:11) (10) and (c) the glycan binding domain of the family 98 glycoside hydrolases from S. pneumoniae (SP2159) (SEQ ID NO:12) (19). The letters above the LLY sequence in panel a represent the amino acid differences with the GenBank sequence of Sm-hPAF (accession number AB051299.1). Homology comparisons were carried out using CLC Free Workbench version 4.6 (CLCBio). The conserved undecapeptide sequence of the CDCs is boxed in panel a. The conserved active site residues of AAA are bolded and underlined in panels b and c. Conserved residues (*), conservative substitutions (:).

FIG. 2. Oligomer formation by LLY. Purified PFO, LLY and LLY^{CDC} were incubated in the absence and presence of cholesterol-rich liposomes and the monomer and oligomer species were separated by SDS-PAGE. Shown is the coomassie stained gel. The smearing of the oligomer bands results from the presence of the liposome derived lipids in the oligomer samples.

FIG. 3. Platelet aggregation by LLY. Shown in (a) are the aggregometer recordings for platelets treated with LLY (71 nM), LLY^{CDC} (95 nM) and LLY^{Lec} (280 nM), and the positive control convulxin (500 ng/ml). Shown in (b) are confocal microscopic images of untreated, LLY treated and convulxin treated platelets. These fields are representative of 12 separate fields.

FIG. 4. Calcein release from LLY-treated platelets. Calcein loaded platelets were assayed by flow cytometry before (shaded peak) and after treatment with 0.7 nM (dashed line) or 1.4 nM (solid line) LLY (a) or LLY^{CDC} (b).

FIG. 5. Platelet lysis requires pore formation. Shown are aggregometer readings for platelets treated with prepore locked mutants of LLY (a), PFO (b) and ILY (c) in their oxidized (LLY^{ppl-ox}, PFO^{ppl-ox} and ILY^{ppl-ox}) and reduced forms (LLY^{ppl-red}, PFO^{ppl-red} ILY^{ppl-red}). Conditions were similar to those in FIG. 3a; all toxins were added at approximately 70 nM. The reduction of the disulfide allows each prepore locked mutant to convert to the pore complex.

FIG. 6. Glycan binding by LLY, LLY^{Lec} and LLY^{Lec-R112A}. Version 3.0 of the printed glycan microarray, containing 320 eukaryotic derived glycans (see Experimental Procedures for a link to the full list of glycans on this array), was probed with fluorescently-tagged (Alexa 488) versions of (a) LLY (670 nM), (b) LLY^{Lec} (560 nM) and (c) LLY^{Lec-R112A} (560 nM) that had been labeled at the cysteine substituted Gln-190. Standard error of the mean is shown as gray error bars. Note: The microarray analyses herein were carried out using the updated version 3.0 of the glycan array whereas the results in Table 3 were obtained using version 2.1. This was a result of a change in the microarray version during the course of these studies. There are only 5 differences in fucose-containing glycans in the two versions. Only two of these glycans, numbers 290 and 301 on the version 3.0 array, contain the H-antigen structure and were shown to be bound by LLY^{Lec} whereas the other three glycans were not (data not shown). In panel (d) the K_{d} of the Le^{y}-LLY^{Lec} interaction was determined by measuring changes in the anisotropy (Δr) of Le^{y} (kept constant at 167 nm) in the presence of LLY^{Lec} that was varied from 300 nM to 120 μM. Le^{x} incubated with the highest concentration of LLY (120 μM) exhibited a Δr of ≈10% of that observed for Le^{y}.

FIGS. 7A & 7B. LLY^{Lec} preferentially binds Le^{y} and Le^{b} glycans. Version 2.1 of the printed glycan microarray (Consortium for Functional Glycomics) was probed with Alexa-488 labeled LLY^{Lec-Q190C} at 560 nM, 56 nM and 5.6 nM. To demonstrate specificity of binding by LLY^{Lec} all glycans that were bound at the highest LLY^{Lec} concentration are shown as well as related glycans that were not bound or weakly bound. The average RFU (from 4-6 analysis with high and low values eliminated) for each bound glycan was determined as described in the Methods and the % CV (100×Standard Deviation÷Average) provides an indication of the precision of each measurement. All other glycans on the version 2.1 array probed at the highest concentration of ILY^{Lec} did not exhibit signals above background. AVG RFU is average relative fluorescence units; SP, spacer arm or linker between glycan and surface of the array; SP0, —CH₂CH₂NH₂; and SP8, CH₂CH₂CH₂NH₂. Circles; galactose, squares; GalNac, triangles, fucose, squares; GlcNAc.

FIG. 8. The glycan-binding domain of LLY modulates pore-forming activity. LLY dependent calcein release from calcein-loaded PRP was measured by flow cytometry. (a) LLY dependent calcein release from platelets of high (donor 1) and low responding donors (donor 3) which were preincubated without (solid lines) or with (dashed lines) LLY^{Lec} (56 nM). No change is observed with LLY^{Lec} alone (not shown). (b) Calcein release from platelets treated with LLY or LLY^{CDC}. (c) Same as the experiments shown in (a) with donor 1 platelets except that LLY^{CDC} was substituted for LLY.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of composition and methodology set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

According to the present invention, conjugates for use in the treatment for or diagnosis of cancer or other epithelial derived diseases are provided. The present invention provides conjugates that include a protein or peptide ligand having the ability to specifically and stably bind to a difucosylated receptor or binding site on an outer surface of a tumor or cancer cell having the receptor, particularly which is expressed or overexpressed on a surface of the tumor or cancer cell). The conjugate comprises an anticancer agent that is operatively attached to the ligand, wherein the anticancer agent preferably is selectively toxic to cancer cells. The ligand portion of the conjugate (the LLY-fucolectin domain or fragment thereof described herein) specifically and stably binds to the external Lewis y or Lewis b glycans on the outer surface of the cell. The anticancer agent and the ligand may be directly coupled together or indirectly coupled together via a linker. In addition, as described below, the anticancer agent may be conjugated to PEG, or the conjugate may be encapsulated in a liposome.

Streptococcus mitis produces a protein previously known as platelet aggregation factor (PAF) that has been determined herein to actually be a cholesterol-dependent cytolysin. The protein of the present invention (having differences from PAF in twelve amino acid positions) is referred to hereinafter as lectinloysin (LLY) (SEQ ID NO: 1) which is encoded by the nucleic acid SEQ ID NO:2. We have also genetically isolated a 162 amino acid fragment of the LLY, referred to herein as LLY-fucolectin domain or LLY^{Lec} (SEQ ID NO:3), determined that (1) it is active as a lectin and (2) that it exhibits a unique specificity for Fucose α1-2 Galβ (i.e., H-antigen, Fucα1-2 Galβ) and glycans that contain this disaccharide. Further, it has been discovered that at high stringency (i.e., at concentrations 10-500 times lower than is used to detect H-antigen containing glycans) LLY-fucolectin domain specifically binds only Lewis b (Le^{b}) and Lewis y (Le^{y}) antigens. Hence it is a novel fucolectin; no other lectin is known to bind with high specificity to the same spectrum of Fucα1-2 Galβ-containing glycans. The gene encoding the novel LLY-fucolectin domain was genetically isolated from the gene for platelet aggregation factor from Streptococcus mitis strain SK597 by amplifying out the coding sequence of the fucolectin and subcloning it into an expression vector.

As described in more detail below, the LLY-fucolectin domain of the invention can be easily modified with various therapeutic agents or recorder probes (e.g., fluorescent probes) without affecting its activity that allows it to be used in a wide array of clinical and research applications. The protein is stable and highly soluble. The LLY-fucolectin domain has been cloned, expressed in E. coli and purified in high quantities, as described herein.

As described in further detail below, a glycan array of 320 glycans was probed using a fluorescently labeled LLY-fucolectin domain of the present invention. The glycan binding activity of the native LLY-fucolectin domain was knocked out by changing the fucose binding site Arg-Gly-Asp sequence (positions 76-78 of SEQ ID NO:3) (“RGD”) to Ala-Gly-Asp. As discussed below, this mutation completely eliminates its binding activity and also shows that the glycan-binding results are due to specific and not non-specific binding of the LLY-fucolectin domain.

As is evident from the glycan array results discussed and shown below the novel LLY-fucolectin domain described herein substantially only binds to glycans that contain the Fucoseα1-2 Galβ in their structure, and has the highest specificity for glycans that contain the type 1 and 2H core glycan structure (H type 1, Fucα1-2Galβ1-3GlcNAcβ; H type 2, Fucα1-2Galβ1-4GlcNAcβ), and has the highest preference for the Lewis b (Le^{b}, Fucα1-2Galβ1-3(Fucα1-4)GlcNAcβ) and Lewis y (Le^{y}, Fucα1-2Galβ1-4(Fucα1-3)GlcNAcβ) antigens. At high concentration (i.e., 50 μg/ml) it will detect most glycans that contain Fucα 1,2Galβ whereas at low concentrations (i.e., 0.1 μg/ml) it preferentially recognizes Le^{y} and Le^{b} type glycans. Based on these data, LLY-fucolectin domain exhibits a unique glycan specificity not found in any other lectin or antibody, previously known.

As utilized in accordance with the present disclosure, the following terms, unless otherwise indicated, shall be understood to have the following meanings:

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a coding sequence isolated away from, or purified free from, unrelated genomic DNA, genes and other coding segments. Included within the term “DNA segment”, are DNA segments and smaller fragments of such segments, and also recombinant vectors, including, for example, plasmids, cosmids, phage, viruses, and the like. In this respect, the term “gene” is used for simplicity to refer to a functional protein-, polypeptide- or peptide-encoding unit. As will be understood by those in the art, this functional term includes genomic sequences, cDNA sequences or combinations thereof. “Isolated substantially away from other coding sequences” means that the gene of interest forms the significant part of the coding region of the DNA segment, and that the DNA segment does not contain other non-relevant large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

Preferably, DNA sequences in accordance with the present invention will further include genetic control regions which allow the expression of the sequence in a selected recombinant host. The genetic control region may be native to the cell from which the gene was isolated, or may be native to the recombinant host cell, or may be an exogenous segment that is compatible with and recognized by the transcriptional machinery of the selected recombinant host cell. Of course, the nature of the control region employed will generally vary depending on the particular use (e.g., cloning host) envisioned.

Truncated genes also fall within the definition of preferred DNA sequences as set forth above. Those of ordinary skill in the art would appreciate that simple amino acid removal can be accomplished, and the truncated versions of the sequence simply have to be checked for the desired biological activity in order to determine if such a truncated sequence is still capable of functioning as required. In certain instances, it may be desired to truncate a gene encoding a protein to remove an undesired biological activity, as described herein.

Nucleic acid segments having a desired biological activity may be isolated by the methods described herein. The term “a sequence essentially as set forth in SEQ ID NO:X” means that the sequence substantially corresponds to a portion of SEQ ID NO:X and has relatively few amino acids or codons encoding amino acids which are not identical to, or a biologically functional equivalent of, the amino acids or codons encoding amino acids of SEQ ID NO:X. The term “biologically functional equivalent” is well understood in the art and is further defined in detail herein, as a gene having a sequence essentially as set forth in SEQ ID NO:X, and that is associated with the ability to perform a desired biological activity in vitro or in vivo.

The art is replete with examples of practitioner's ability to make structural changes to a nucleic acid segment (i.e. encoding conserved or semi-conserved amino acid substitutions) and still preserve its enzymatic or functional activity when expressed. See for example (52, 53, 54).

These references and countless others, indicate that one of ordinary skill in the art, given a nucleic acid sequence or an amino acid or an amino acid sequence, could make substitutions and changes to the nucleic acid sequence without changing its functionality. One of ordinary skill in the art, given the present specification, would be able to identify, isolate, create, and test DNA sequences and/or enzymes that produce natural or chimeric or hybrid molecules having a desired biological activity. As such, the presently claimed and disclosed invention should not be regarded as being solely limited to the specific sequences disclosed herein. Examples of such standardized and accepted functionally equivalent amino acid substitutions are presented in Table 1.

TABLE 1

| | Conservative and Semi- |
|---|---|
| Amino Acid Group | Conservative Substitutions |
| Nonpolar R Groups | Alanine, Valine, Leucine, Isoleucine, |
| | V Methionine, Phenylalanine, |
| | Tryptophan |
| Polar, but uncharged, R Groups | Glycine, Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

As will be appreciated by a person of ordinary skill in the art, modifications may be made to the LLY-fucolectin domain or fragments thereof which are used in the conjugates of the invention. Types of polypeptide modification may comprise for example, amino acid insertions (i.e., addition), deletions and substitutions (i.e., replacement), either conservative or non-conservative (e.g., D-amino acids, desamino acids) in the polypeptide sequence where such changes do not substantially alter the overall biological activity of the polypeptide (Lewis y/Lewis b binding). Polypeptides of the present invention may comprise for example, biologically active mutants, variants, fragments, chimeras, and analogs; fragments encompass amino acid sequences having truncations of one or more amino acids, wherein the truncation may originate from the amino terminus (N-terminus), or the carboxy terminus (C-terminus). Analogs of the invention may involve an insertion, deletion, or a substitution of one or more amino acids.

Examples of substitutions include conservative substitutions (i.e., wherein a residue is replaced by another of the same general type as shown in Table 1). As is understood, naturally occurring amino acids may be sub-classified as acidic, basic, neutral and polar, or neutral and non-polar. In some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable.

The DNA segments of the present invention encompass DNA segments encoding biologically functional equivalent proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to antigenicity of the protein or to test mutants in order to examine biological activity at the molecular level or to produce mutants having changed or novel enzymatic activity and/or substrate specificity.

By “polypeptide” is meant a molecule comprising a series of amino acids linked through amide linkages along the alpha carbon backbone. Modifications of the peptide side chains may be present, along with glycosylations, hydroxylations and the like. Additionally, other nonpeptide molecules, including lipids and small molecule agents, may be attached to the polypeptide.

Another preferred embodiment of the present invention is a purified nucleic acid segment that encodes a protein in accordance with the present invention, further defined as being contained within a recombinant vector. As used herein, the term “vector” or “recombinant vector” refers to a vector that has been modified to contain a nucleic acid segment that encodes a desired protein or fragment thereof. The recombinant vector may be further defined as an expression vector comprising a promoter operatively linked to said nucleic acid segment.

A further preferred embodiment of the present invention is a host cell, made recombinant with a recombinant vector comprising one or more genes encoding one or more desired proteins, such as a conjugate. The preferred recombinant host cell may be a prokaryotic cell. In another embodiment, the recombinant host cell is an eukaryotic cell. As used herein, the term “engineered” or “recombinant” cell is intended to refer to a cell into which one or more recombinant genes have been introduced mechanically or by the hand of man. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter associated or not naturally associated with the particular introduced gene.

In preferred embodiments, the DNA segments further include DNA sequences, known in the art functionally as origins of replication or “replicons”, which allow replication of contiguous sequences by the particular host. Such origins allow the preparation of extrachromosomally localized and replicating chimeric or hybrid segments of plasmids, to which the desired DNA sequences are ligated. In more preferred instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The nucleic acid segments of the present invention, regardless of the length of the coding sequence itself, may be combined with other DNA sequences, such as promoters, polyadenylation signals, additional restriction enzyme sites, multiple cloning sites, epitope tags, polyhistidine regions, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

The term “effective amount” refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example but is not limited to, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease in conjunction with the conjugates of the present invention. This concurrent therapy can be sequential therapy where the patient is treated first with one drug and then the other, or the two drugs are given simultaneously.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The terms “cancer” and “cancerous” refer to or describe the physiological condition in mammals that is typically characterized by unregulated cell growth.

The term patient includes human and veterinary subjects. “Mammal” for purposes of treatment refers to any animal classified as a mammal, including human, domestic and farm animals, nonhuman primates, and any other animal that has mammary tissue.

In summary, in one embodiment, described herein is a LLY-fucolectin domain moiety and shown that (1) the LLY is a functional cholesterol-dependent cytolysin, (2) that the amino terminal sequence of LLY is a functional LLY-fucolectin domain, and (3) that the LLY-fucolectin domain exhibits a unique glycan specificity for H-antigen (glycans with Fucα1-2Galβ) and at high stringency for Lewis b and Lewis y antigens.

### Experimental

Described herein are properties of a purified protein, similar to that known as “Platelet Aggregation Factor” (PAF), which is referred to herein as lectinolysin or LLY hereafter, derived from Streptococcus mitis SK597. These studies show that LLY is a pore-forming CDC with a functional fucose-binding domain as described above. LLY does not trigger platelet aggregation, but does form pores in platelet membranes. The isolated fucose-binding lectin domain of LLY (“LLY-fucolectin domain”) exhibits specificity for glycans containing the Lewis y (Le^{y}) and b (Le^{b}) antigens as noted elsewhere herein. The lectin domain of LLY appears to modulate the pore-forming activity of LLY in a glycan-dependent manner after LLY binds to the cell surface.

Bacterial strains, plasmids, and chemicals. The full-length gene for LLY was cloned by PCR from the chromosomal DNA of S. mitis strain SK597 using the primers ATGAATCAAGAAAAACGTTTGCATCGCTTTGTCAAAAAG (SEQ ID NO:5) and the reverse primer sequence 5′-TTACTCATTCACAATTTTTTCATCAACTTTAGGGTTTAG (SEQ ID NO:6). For purposes of expression the cloned LLY gene was amplified with the primers 5′-TCGGATCCGAGCAAGGGAATCGTCCAGTTG (SEQ ID NO:7) that introduced a 5′ BAMH1 endonuclease site and 5′-CTGAATTCTTACTCATTCACAATTTTTTCATCAACTTTAGGG (SEQ ID NO:8) that placed a 3′ EcoR1 endonuclease after the stop codon at the 3′ end of the gene. These primers removed the signal peptide and placed the gene for the secreted LLY in-frame in the pTrcHisA vector (Invitrogen). ILY and PFO were previously cloned into pTrcHisA as described (12, 13). All mutations were made in the native LLY (naturally cysteine-less), ILY (naturally cysteine-less) or cysteine-less PFO (PFO^{C459A}) background. All chemicals and enzymes were obtained from Sigma, VWR or Research Organics. All fluorescent probes were obtained from Molecular Probes (Invitrogen).

Generation and purification of recombinant LLY, ILY and PFO and Derivatives. Using PCR QuikChange mutagenesis (Stratagene), various amino acid substitutions were introduced into the native LLY, PFO or PFO^{C459A}. For example the glutamine at position 190 of LLY, (position 154 of the isolated fucolectin domain) was substituted with cysteine. The Oklahoma Medical Research Foundation Core DNA Sequencing Facility performed all DNA sequence analysis. The expression and purification of recombinant LLY, ILY, PFO and their derivatives from Escherichia coli were performed as described (13, 14). Purified protein was dialyzed into buffer [300 mM NaCl, 10 mM MES, 1 mM EDTA; (pH 6.5)] overnight at 4° C. made 5 mM in dithiothreitol (DTT) and 10% (v/v) sterile glycerol and stored at −80° C. For mutants that contained an engineered disulfide bridge the DTT was left out of the storage buffer.

PCR screening streptococci for the LLY gene. Primers were used in the PCR amplification at a 20-pmol final concentration. These primers were designed to amplify out the entire coding sequence for the LLY gene. The forward primer sequence was SEQ ID NO:5 and the reverse primer sequence was SEQ ID NO:6. Chromosomal DNA from each streptococcal strain was prepared using the Invitrogen Easy DNA Kit and 100 ng was added to PCR reaction. An additional 2 μl of 25 mM MgCl₂ was added to Qiagen Master Mix for a final PCR volume of 25 μl. Each PCR product was separated on a 1% agarose gel.

Chemical modification of LLY and its derivatives with sulfhydryl-specific fluorescent probes. Cysteine derivatives of LLY and LLY^{Lec} (i.e., the 162 amino acid fucolectin domain) were modified with Alexa Fluor 488 C₅-maleimide (Invitrogen) via the sulfhydryl group as previously described (12). Modified protein stocks were adjusted to 10% (v/v) sterile glycerol, quick frozen in liquid nitrogen, and stored at −80° C. Proteins were labeled at an efficiency of 75% or greater.

K_{d} determination. Fluorescein-labeled glycans were prepared as previously described using free reducing oligosaccharides (15). The K_{d} of the interaction of LLY^{Lec} with Le^{y} was determined by fluorescence anisotropy. Fluorescence measurements were carried out using an SLM 8100 fluorimeter equipped with excitation and emission calcite polarizers. The excitation and emission monochrometer wavelengths were set to 480 nm and 520 nm, respectively. The excitation and emission bandpass was set to 4 nm. Sample volume was maintained at 300 μl in a Starna microcell with a total capacity of 560 μl. Temperature was maintained at 23° C. The fluorescein labeled Le^{y} was maintained at 167 nM and the LLY^{Lec} was varied from 300 nM to 120 μM. The anisotropy (r) was calculated at each concentration of LLY from the equation shown below.

$$r = \frac{{I}_{W} - {GI}_{VH}}{{I}_{W} + 2 {GI}_{VH}}$$

I is intensity, V is vertically polarized light and H is horizontally polarized light. The G factor (G=I_{HV}/I_{HH}) was calculated for each sample. The Δr was plotted versus the concentration of LLY^{Lec} and the dissociation constant (K_{d}) was calculated using the equation Y=Bmax*X/(Kd+X) for one site binding where Bmax is the maximal binding and the K_{d} is the concentration of ligand (in this case the LLY^{Lec}) necessary to achieve half-maximal binding. Data analysis was carried out using Graphpad Prism software.

Platelet aggregation assay. Platelet rich plasma (PRP) was prepared from normal human donors as described previously (16). Blood donors were free of aspirin or other anti-platelet medications for at lease seven days prior to donation. Aggregation experiments were performed with a Model PAP-4 aggregometer from Bio/Data Corporation. Total reaction volume was 200 μl. Convulxin-induced aggregation (500 ng/ml final concentration, (16)) was used as a positive control. LLY was assayed at 71 nM, LLY^{CDC} at 95 nM and LLY^{Lec} at 280 nM.

Calcein release assay. PRP was diluted 1:100 with HEPES/saline assay buffer (2 mM CaCl₂, 1 mM MgCl₂, 150 mM NaCl, 10 mM HEPES, pH 7.5) and incubated with 2 μM calcein-acetoxy methyl ester (Molecular Probes, Eugene, Oreg.) for 10 minutes at room temperature as previously described (17). LLY (0.7-2.9 nM) was incubated with the calcein-loaded PRP for 10 minutes at room temperature. Samples were then diluted with HEPES/saline and analyzed on a FACSCalibur flow cytometer (Becton-Dickinson) using settings as described previously (17). Cells losing calcein fluorescence were quantified as a percentage of total platelets.

The EC₅₀ values for LLY- and LLY^{CDC}-dependent calcein release were calculated using a nonlinear sigmoidal dose-response curve fit of the data (Prism Software).

Glycan microarray binding assay and scanning. Glycan microarrays were prepared as described previously (33,37) and obtained from the NIH/NIGMS-funded Consortium for Functional Glycomics (see http://www.functionalglycomics.org/static/index.shtml). Before assay, the slides were rehydrated for 5 minutes in TSM buffer (20 mM Tris-HCL, 150 mM sodium chloride (NaCl), 0.2 mM calcium chloride (CaCl₂) and 0.2 mM magnesium chloride (MgCl₂). Alexa-488-labeled LLY^{Q190C} and its derivatives were used in the binding assay (560-640 nM), and the bound proteins were detected directly via the fluorescent tag.

The slides were scanned with a Perkin Elmer ProScanarray MicroArray Scanner (Waltham, Mass.) using an excitation wavelength of 488 nm. ImaGene software (BioDiscovery, Inc., El Segundo, Calif.) was used to quantify fluorescence. The data are reported as average relative fluorescence units (RFU) from 4 of the 6 replicates (after removal of the highest and lowest values) for each glycan represented on the array. Versions 2.1 and 3.0 of the printed array were used in these studies and a complete list of glycans printed on these arrays can be found at the Functional Glycomics Gateway website:

www.functionalglycomics.org/static/consortium/resources/resourcecoreh10.shtml.

SDS-agarose gel electrophoresis (SDS-AGE). SDS-AGE of monomer and oligomer species of the various CDCs was carried out as previously described (18).

Liposome preparation. Cholesterol-phosphatidylcholine (55/45 mol %) liposomes were prepared as previously described (13). Lipids and cholesterol were obtained from Avanti Polar Lipids.

### Results

The Primary Structure of LLY

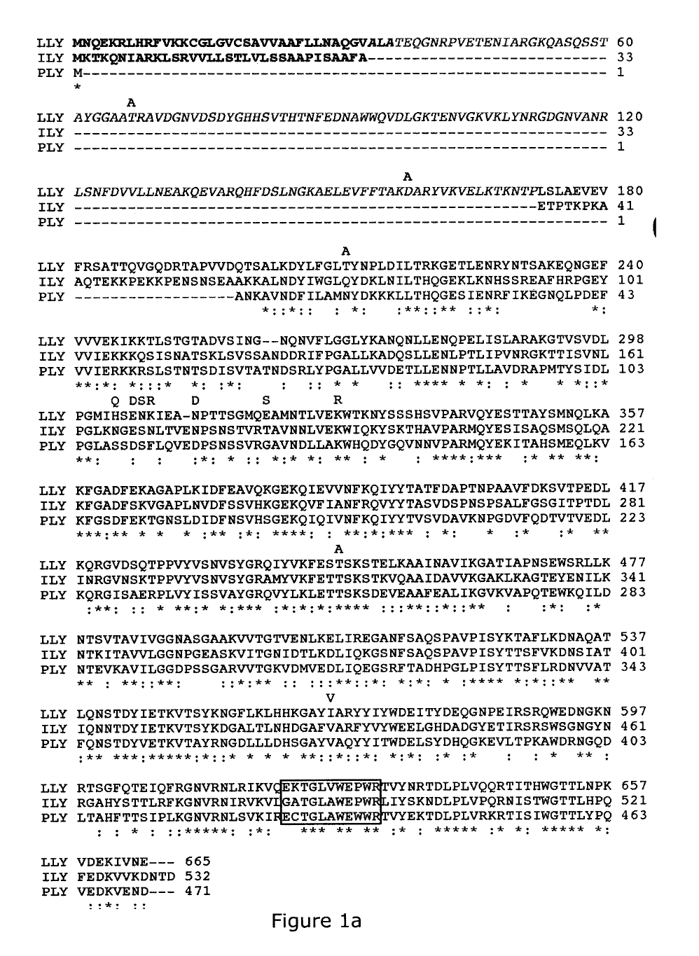

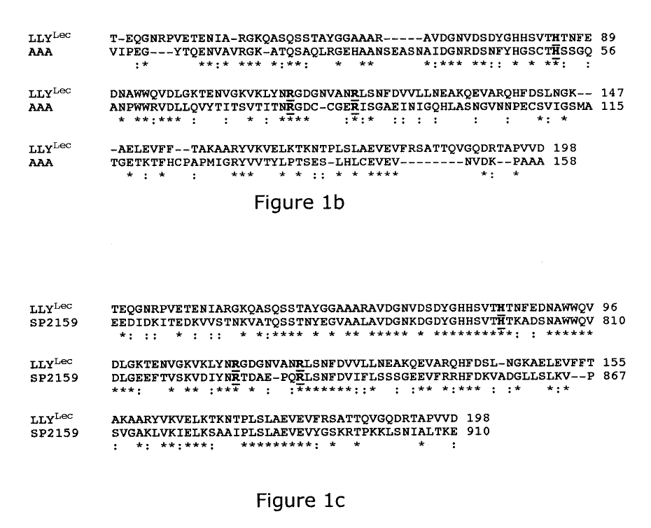

The LLY gene was cloned from S. mitis SK597, which exhibits characteristics of both S. mitis and S. oralis (Hollingshead, unpublished data). The gene sequence (SEQ ID NO:2; also see GenBank Accn. No. EU597013) exhibits the typical AT-rich (60% A-T) sequence of streptococcal genes. The deduced primary structure of LLY is nearly identical to the GenBank sequence for Sm-hPAF from S. mitis Nm-65 (accession number AB051299.1), wherein twelve mostly conservative amino acid differences were identified (FIG. 1a). These differences did not result from PCR-generated errors in the LLY sequence reported herein; the same sequence was derived from the cloned products of two separate PCR amplification experiments. Hence, the primary structure of LLY can apparently vary to some extent. The open reading frame encodes a protein with a mass of 73,780.3 d. Based on the previously published amino terminal sequence of purified protein from the supernatant of S. mitis NM-65 (7) the amino terminus of secreted LLY is located at or near Thr-37 which is preceded by a 36 residue peptide that exhibits a typical Type II signal peptide structure. The primary structure of LLY from residue 199 to 665 (FIG. 1a) is most closely related to CDCs from Streptococcus intermedius (ILY) (SEQ ID NO:9), and Streptococcus pneumoniae (PLY) (SEQ ID NO:10). The CDC structure of LLY also contains an undecapeptide sequence (EKTGLVWEPWR) (SEQ ID NO:13) that is similar to the hallmark consensus sequence (ECTGLAWEWWR) (SEQ ID NO:14) found in most CDCs, but contains substitutions at positions (underlined) similar to those found in ILY (GATGLAWEPWR) (SEQ ID NO:15).

Unlike other characterized CDCs, LLY contains a fucolectin domain (SEQ ID NO:3) fused to its amino terminus (excluding the signal peptide) (FIG. 1a). This peptide region exhibits similarity with the Anguilla anguilla (European eel) agglutinin (AAA) (33% identity) (10) (FIG. 1b) and the carboxy-terminal carbohydrate-binding module of a putative member of the family 98 glycoside hydrolases from S. pneumoniae (47% identity) (FIG. 1c) (19). Both proteins are fucose-binding proteins that exhibit a preference for glycans containing the H-antigen structure (Fucα1-2Galβ1-3(4)GlcNAc-R).

Distribution of the LLY Gene

A collection of 165 strains of mostly mitis group streptococci was examined for the presence of the LLY gene by PCR using primers that encompassed its complete coding region. From this collection, twelve S. mitis strains, one S. pneumoniae, four S. pseudopneumoniae and three S. mitis/oralis strains carried the gene. The PCR product size did not vary in size for any of the positive strains and co-migrated with the 2 Kb marker, consistent with its 1998 base pair sequence (including the stop codon) (data not shown). We confirmed expression and secretion of LLY in the SK597 strain (data not shown) that served as the source of the gene for our studies, but have not confirmed its expression in the other species and strains that appear to carry the gene.

Hemolytic Activity of LLY and Derivatives

The coding region for LLY was cloned, expressed in E. coli and purified (see methods). Shown in Table 2 is the HD50 (dose of toxin required for 50% hemolysis under standard conditions, see Methods) for purified LLY, LLY^{CDC} (LLY lacking the amino terminal signal peptide and lectin binding domain), PFO and ILY. In Table 2 are the HD50 values, using human erythrocytes, for LLY and LLY^{CDC}, and for comparison those of PFO and ILY. All of the CDCs exhibit picomolar HD50 values, but it appears LLY and LLY^{CDC} are 4-5 fold less active than PFO and ILY. These values are within the typical range for these toxins and can vary depending on the cell type and species of origin. As described below, however, the amino terminal lectin domain appears to exert a glycan-dependent enhancement of LLY activity with platelets.

TABLE 2

| CDC | HD₅₀ (pM) |
|---|---|
| ILY | 1.1 |
| PFO | 0.8 |
| LLY | 4.1 |
| LLY^{CDC} | 4.4 |

Table 2. Hemolytic activity of LLY and related CDCs used in this study. The relative HD₅₀ (hemolytic dose for 50% lysis of a standard erytheocyte suspension, see Methods) for each CDC used in these studies is provided.

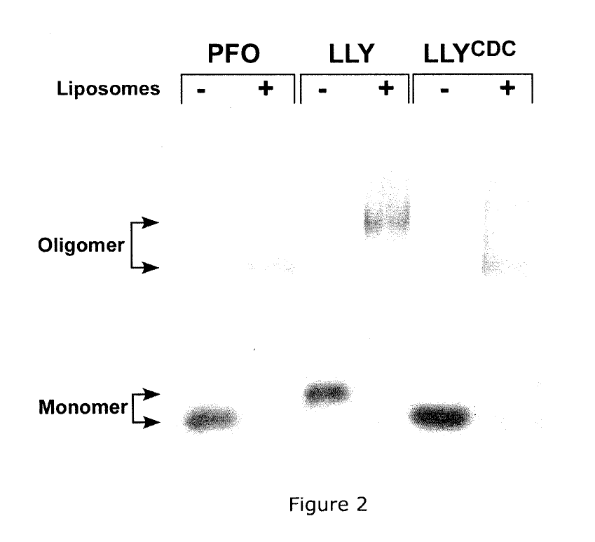

Similar to other CDCs, LLY and LLY^{CDC} also formed large oligomeric complexes on cholesterol-rich liposomes (FIG. 2). LLY^{CDC} monomer (52,147 Da) and oligomer migrated similarly to PFO monomer (52,672 Da) and oligomer. The similarity in the mass of both proteins suggests that both form oligomers with similar numbers of monomers. In contrast to PFO and LLY^{CDC} the migration of the LLY oligomer was significantly retarded. The slower migration of LLY was likely due to the additional mass contributed by the amino terminal lectin domain (17,751 Da). The CDCs typically form oligomers of 35-40 monomers (20), thus the oligomer mass of LLY would be at least 600 kDa greater than that of LLY^{CDC} or PFO, consistent with its slower migration on the gel.

LLY and Other CDCs do not Aggregate Platelet

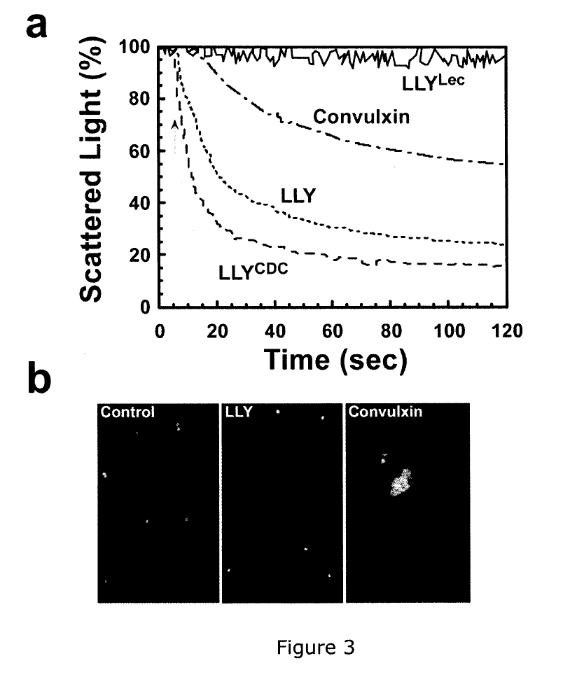

Ohkuni, et al. (7) reported that LLY (Sm-hPAF) stimulated platelet aggregation in human platelet rich plasma (PRP). Qualitative analysis of platelet aggregation is commonly determined using an aggregometer, an instrument that measures the decrease in the light scattering properties of PRP as platelets aggregate into large complexes. Aggregometer analysis herein also showed apparent LLY-dependent platelet aggregation resulting in the decreased light scatter by the platelets (FIG. 3a). Similar results were observed with purified LLY^{CDC}, demonstrating that the predicted amino terminal fucose-binding lectin domain was not required for this activity (FIG. 3a). Purified lectin domain (LLY^{LEC}) did not induce any change in the light scattering properties of the platelets (FIG. 3a). To confirm the aggregometer measurements reflected platelet aggregation by LLY and LLY^{CDC} we performed a microscopic examination of the platelets after treatment. Surprisingly, we could not identify any aggregated platelets (FIG. 3b). By comparison, we observed large platelet aggregates in PRP treated with convulxin, a nonenzymatic glycoprotein from venom of Crotalus durissus terrificus that induces platelet aggregation. Although the aggregometer readings indicated that LLY and LLY^{CDC} could aggregate platelets the microscopic examination did not support this interpretation. Although unlikely, it was possible that we simply missed the aggregates formed by LLY. Therefore we quantified the relative levels of platelets in each preparation.

To measure the relative levels of platelets present in these mixtures we utilized a flow cytometry based approach. PRP was either left untreated or treated in the aggregometer with 140 nM LLY or 500 ng/ml convulxin for 3 minutes at 37° C. with stirring. Aliquots of treated PRP were then incubated with FITC-labeled anti-glycoprotein IIb/IIIa antibody and platelet concentration was determined by flow cytometry. The relative platelet concentration in each sample was determined by the time required to accumulate 5,000 events on the cytometer. If platelets aggregated into larger complexes it would take longer to accumulate 5000 counts. If no significant aggregation occurs then the time required for 5000 counts should be similar to untreated platelets. The LLY-treated and control (untreated) samples had nearly identical platelet concentrations (5,000 counts/34 sec and 5,000 counts/31 sec, respectively). In contrast, the convulxin-treated sample was significantly depleted of individual platelets (5,000 counts/84 sec). Hence, both convulxin and LLY induce changes in the light scattering properties of the platelets, but it is clear that LLY does not induce platelet aggregation. How then did LLY affect the light scattering properties of the platelets without aggregation?

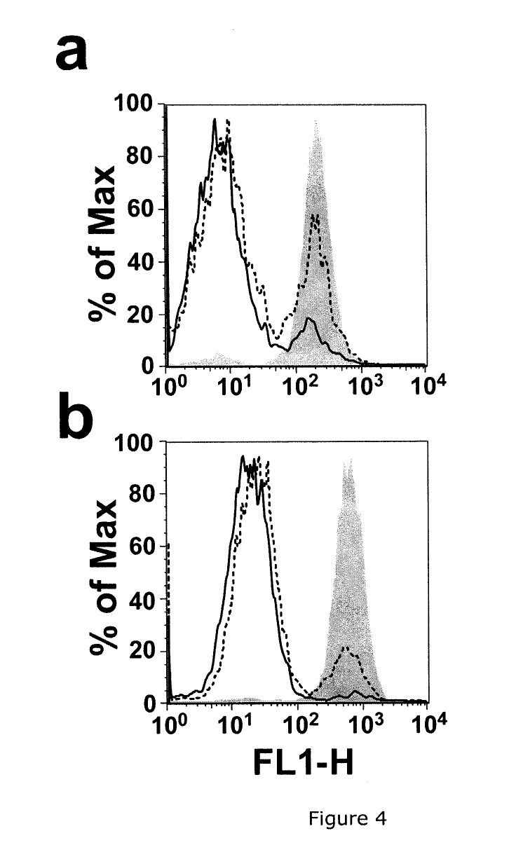

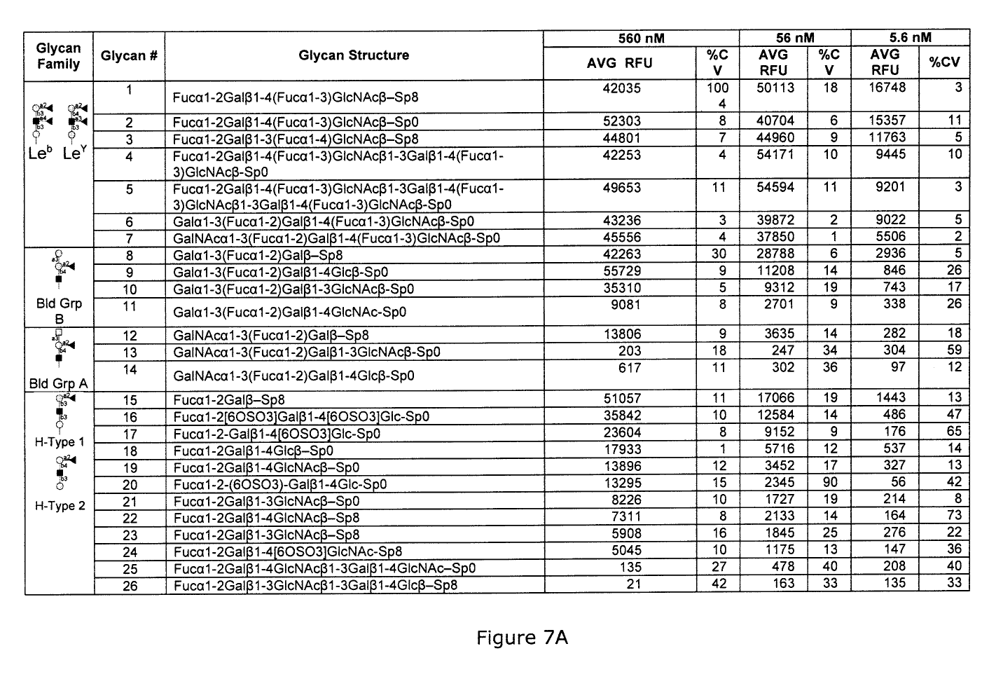

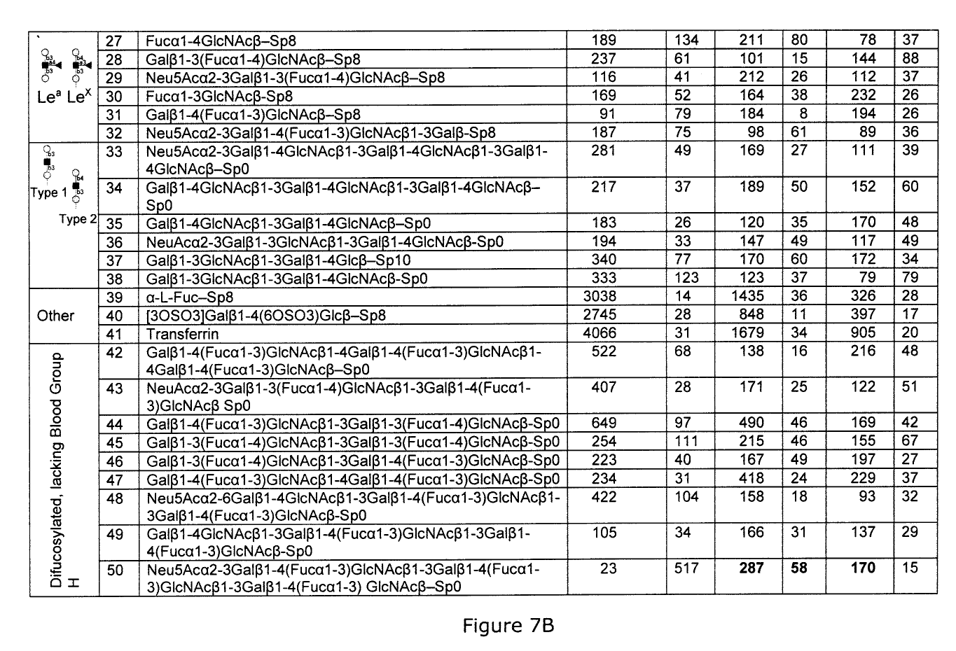

The light scattering properties of cells often results from shape change and/or loss of cytoplasmic contents. For example, erythrocytes lose their light scattering properties upon pore formation by the CDC PFO (21). Pore formation in platelets was determined by monitoring calcein release from LLY treated platelets. As shown in FIG. 4, calcein is efficiently released in a dose dependent manner from the platelets treated with LLY or with LLY^{CDC}, suggesting that each formed a membrane pore. The relative EC₅₀ for calcein release by both proteins was quantified below in the studies described in FIG. 7 where we show that LLY exhibits variable activity on platelets from different donors.

To assess whether the observed change in light scattering properties of LLY in the aggregometer was dependent on pore formation, a mutant of LLY was trapped in the prepore complex by the introduction of an engineered disulfide, but which could be converted to the pore-forming oligomer by reduction of this bond. We previously showed a disulfide bridge between domains 2 and 3 of the CDC structure prevents membrane insertion of the transmembrane β-hairpins, but not binding and assembly of the prepore oligomer (22). When the disulfide bridge was reduced the transmembrane β-hairpins inserted into the membrane and formed the pore.

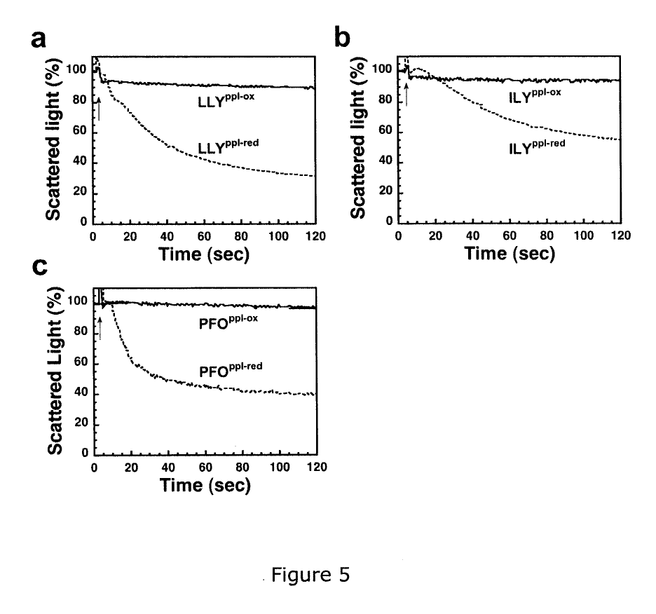

A prepore locked mutant of LLY was generated by introducing cysteines for Gly-222 and Asn-351 (LLY^{G222C/N351C}), residues analogous to Gly-β3 and Ser-217 of ILY, and Gly-57 and Ser-190 of PFO, which were shown previously to form a disulfide and trap both toxins in prepore complexes [(22) and Soltani and Tweten, unpublished data]. LLY^{G222C/N351C} functioned as expected; when the disulfide bond was oxidized it was not hemolytic, whereas reduction of the disulfide restored near native hemolytic activity (data not shown). Treatment of PRP with oxidized LLY^{G222C/N351C} did not elicit a change in the light scattering properties of the platelets (FIG. 5a), but reduction of the disulfide bond restored this activity. Similarly, the oxidized disulfide locked variants of ILY (FIG. 5b) and PFO (FIG. 5c) did not change the light scattering properties of the platelets, whereas the reduced forms of each triggered significant changes. These data show that the changes in light scattering properties of platelets are dependent on the pore-forming properties of LLY, PFO and ILY and are not the result of platelet aggregation.

Glycan Specificity and Affinity of the LLY Lectin Domain

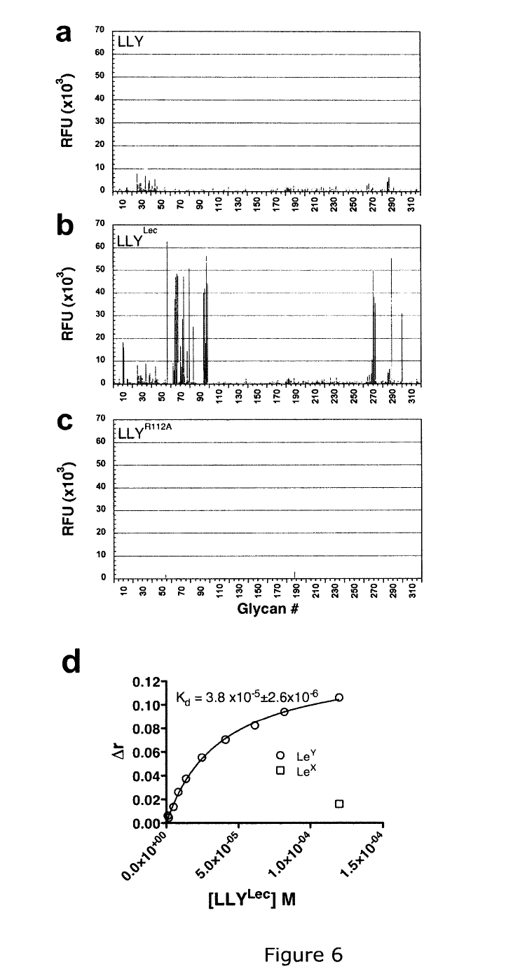

We next determined if the putative fucose-binding lectin domain of LLY was functional. A glycan microarray of vertebrate-type glycans (see Experimental Procedures for a link to the complete list of glycans on this array) was probed with fluorescently tagged LLY. The probe consisted of the cysteine-substituted mutant LLY^{Q190C} labeled with the maleimide derivative of Alexa-488 via the cysteine sulfhydryl (LLY^{Q190C-Alexa}). Gln-190 (position 154 in the isolated fucolectin domain) was chosen for substitution with cysteine since it is located near the junction of the CDC and lectin domains. When the glycan microarray was probed LLY^{Q190C-Alexa} no significant binding was detected (FIG. 6a). This result suggested several possibilities: the lectin domain might bind to glycans that were not represented in the array; the lectin domain was not functional; or the glycan-binding site was sterically occluded or somehow inhibited in the soluble monomer of LLY.

We first tested the latter possibility, primarily because of the amino terminal location of the lectin domain. This location positions the lectin domain distal to the carboxy-terminal domain 4, which contains the membrane-binding site of the CDCs (23-26). If the glycan-binding site was exposed in the monomer, it could bind LLY to cells in an unfavorable orientation, preventing critical domain 4 membrane interactions that initiate a cascade of ordered structural changes in the CDCs that lead to pore formation. Therefore, in this scenario occlusion or inhibition of the glycan-binding site may be necessary until after LLY has bound to the membrane. If correct, then expression of the lectin domain alone should restore its binding activity. A pair of stop codons was introduced after residue 190 of LLY^{Q190C} (residue 154 of the isolated fucolectin) to eliminate the translation of the downstream CDC structure. This construct was designated LLY^{Lec-Q190C}.

Purified LLY^{Lec-Q190C} was fluorescently labeled with Alexa-488 and used to probe the glycan microarray. As shown in FIG. 6b, we observed significant binding to several glycans on the microarray. We also determined whether binding to the glycans on the glycan microarray was lost if we changed a residue that has been shown to be conserved in the fucose binding site of related lectins. The AAA glycan binding site contains three key residues that make polar contacts with the fucose molecule. The Nε of His-52 contacts O-5 of the fucose ring whereas the guanidinium groups of Arg-79 and Arg-86 make contact with 3-OH and 4-OH ring hydroxyls of fucose (10). These residues are also conserved in the fucose-binding site of the family 98 glycoside hydrolases from S. pneumoniae (SP2159) (19) and correspond to residues His-85, Arg-112 and Arg-120 of LLY, respectively (FIGS. 1b and 1c). Arg-112 of LLY^{Lec} (position 76 of the isolated fucolectin domain) was changed to Ala in LLY^{Lec-Q190C}. This mutant (LLY^{Lec-Q190C/R112A}) was labeled with Alexa-488 at Cys190 and used to probe the glycan microarray. This mutation eliminated the glycan binding activity of LLY^{Lec} (FIG. 6c) consistent with the conservation of this active site residue.

We further investigated the binding of LLY^{Lec} to the glycan microarray to determine its glycan specificity. We probed the microarray with three different concentrations of LLY^{Lec} that spanned a 100-fold range of concentration from 5.6 nM to 560 nM. These results (shown in FIGS. 7A and 7B) are shown for all glycans bound at the highest concentration of LLY^{Lec} and selected glycans representative of those not bound by LLY^{Lec} at the highest concentration of LLY^{Lec} (representative of most glycans on the array). When the array was probed at 560 nM LLY^{Lec}, it bound to glycans containing the H-antigen structure (Fucα1-2Galβ1-3(4)GlcNAc-R) as found within the A, B, O, Le^{y} and Le^{b} blood group antigens (glycan ID #s 1-24) and showed little affinity for the non-fucosylated type 1 and type 2 oligosaccharides (glycan ID #s 33-38), fucosylated glycans that did not contain the H-antigen, including Le^{a} and Le^{x} containing structures (glycan ID #s 27-32) and the difucoslyated structures (glycan ID #s 42-50). At this high concentration of LLY^{Lec}, we observed some weak binding to irrelevant glycans (glycan ID # 39-41). However, when the microarray was probed with 10-fold (56 mM) and 100-fold lower (5.6 nM) concentrations of LLY^{Lec}, we observed a marked shift in binding preference for glycans containing Le^{y} and Le^{b} antigens (glycan ID #s 1-7). These results demonstrate that LLY^{Lec} has a unique recognition of difucosylated glycans Fucα1-2Galβ1-3/4(Fucα1-4/3)GlcNAc-R) as found in Le^{b} and Le^{y} blood group antigens. Interestingly, glycans #6 and #7 are such difucosylated glycans, which also contain the blood group B and A determinants, respectively. However, as noted above, when the array is probed at the lowest concentration of LLY^{Lec} it binds weakly or not at all to blood group B (Galα1-3[Fucα1-2]Galβ1-3/4R), blood group A (GaNAclα1-3[Fucα1-2]Galβ1-3/4R) or blood group H (Fucα1-2Galβ) determinants that lack the necessary difucosylated structures.

The affinity of LLY^{Lec} for Le^{y} was determined by measuring changes in fluorescence anisotropy (Δr) of fluorescently tagged Le^{y} as the concentration of LLY^{Lec} was varied. From this analysis the K_{d} of the LLY^{Lec}-Le^{y} interaction was determined to be approximately 38 μM (FIG. 6d). As expected from the microarray data, Le^{x} antigen, which lacks the H-antigen structure, was not bound significantly (<10% of Le^{y}), at the highest concentration of LLY^{Lec}.

The Lectin Domain of LLY Modulates its Pore-Forming Activity

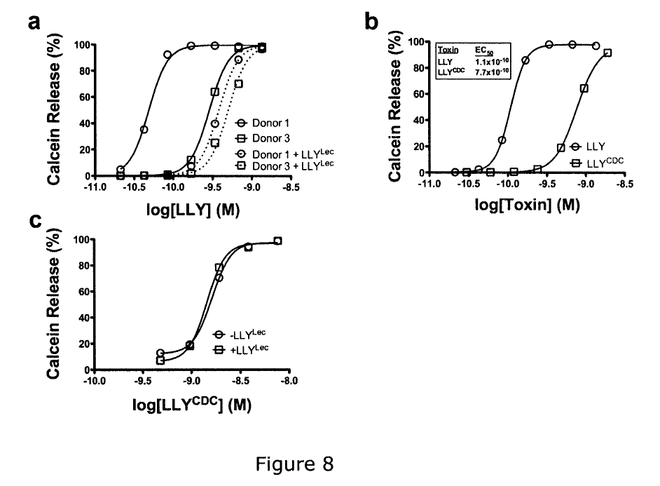

Ohkuni et al. (7) previously showed that platelets of some donors were unresponsive to Sm-hPAF mediated aggregation (shown herein to be due to pore-dependent changes in platelet light scattering properties). This observation suggested that different donor platelets might exhibit different susceptibilities to the pore-forming activity of LLY. Consistent with this prediction, we observed that platelets from different donors exhibited differential susceptibility to LLY (Dale and Friese, unpublished observations). Shown in FIG. 8a is the EC₅₀ for LLY pore formation determined on high (donor 1) and low (donor 3) sensitivity platelets. The platelets from donor 1 are nearly 6-fold more sensitive to LLY (EC₅₀=4.890×10⁻¹¹) than those from donor 3 (EC₅₀=2.88×10⁻¹⁰). These results suggested that a feature of donor 1 platelets significantly enhanced their susceptibility to LLY.

We hypothesized that enhancement of LLY activity resulted from a glycan-dependent interaction of the lectin domain after monomer bound the cell surface. If correct then preincubating platelets with LLY^{Lec} should prevent interaction of the lectin domain of LLY with surface glycans and block the enhancement of LLY activity on the highly sensitive donor 1 platelets. The EC₅₀ for calcein release by LLY from platelets pretreated with LLY^{Lec} (EC₅₀=3.8×10⁻¹⁰) was approximately 8-fold higher than the EC₅₀ for LLY on platelets that were not pretreated with LLY^{Lec} (EC₅₀=4.890×10⁻¹¹) (FIG. 8a). Similar results were observed if the platelets were incubated with 50 mM fucose instead of LLY^{Lec}, but not with 50 mM sucrose as a control (data not shown). Furthermore, pretreatment of the low sensitivity donor 3 platelets with LLY^{Lec} had little impact on the activity of LLY suggesting that the glycan(s) was largely absent from this donor's platelets. The results show the lectin domain increases LLY specific activity, apparently by interacting with one or more glycans on the cell surface of platelets.

These data also predict LLY^{CDC}, which lacks the lectin domain, should be less active than LLY on donor 1's platelets. To test this prediction the EC₅₀ for calcein release by LLY and LLY^{CDC} was determined. As is shown in FIG. 8b the EC₅₀ for LLY^{CDC} on donor 1 platelets was 7-fold higher than the EC₅₀ for LLY, consistent with the change observed in LLY activity on LLY^{Lec} pretreated donor 1 platelets. Furthermore, LLY^{CDC} platelet pore-forming activity was not affected by preincubating the platelets with LLY^{Lec} (FIG. 8c).

This work shows LLY is a functional CDC with an amino terminal fucose-binding lectin domain. The LLY gene was detected in various isolates of the mitis group of the streptococci. LLY exhibits the typical large oligomeric membrane complexes and pore-forming activity of the CDCs. Pore-formation in the platelet membrane was shown herein to be responsible for changes in their light scattering properties rather than the previously reported aggregation (7). The primary structure of LLY is distinct from other CDCs due to the presence of the amino terminal fucose-binding lectin domain (LLY-fucolectin domain). The isolated lectin domain binds to glycans that contain H-antigen structure (Fucα1-2Galβ1-3(4)GlcNAc-R), with a marked preference for the difucosylated Le^{y} and Le^{b} antigens. The glycan binding site is occluded in the soluble complete LLY monomer, but is apparently exposed after the monomer binds to the cell surface since it enhances LLY-dependent pore-forming activity through a glycan-dependent mechanism. To date LLY is the only characterized CDC to exhibit a glycan-binding lectin domain.

The glycan-binding site is occluded in the soluble LLY monomer, presumably to prevent nonproductive interactions that would inhibit the assembly of the oligomeric pore complex. Previous studies showed that the CDC monomer is bound in a perpendicular orientation to the membrane surface via loops at the tip of domain 4, an interaction that initiates ordered conformational changes in the CDC structure necessary for pore formation (reviewed in (27)). The amino terminal location of the lectin domain places it next to domain 1 of the CDC structure and distal to the membrane-binding site in domain 4. If the glycan-binding site was exposed in the soluble monomer its interaction with a surface glycan could bind LLY to the surface in a nonproductive orientation that would prevent the assembly of the oligomeric pore complex. Yet, as described below, the glycan-binding site must be exposed after LLY binds to the membrane surface, since it enhances pore-forming activity.

The molecular models suggest a possible explanation for the occlusion of the glycan-binding site in LLY. The structural model of the LLY^{Lec}, based on the crystal structure of the Le^{y}-binding module of S. pneumoniae family 98 glycoside hydrolase (19), positions the C-terminus of LLY^{Lec} on the same face as Arg-112, a residue we showed herein is conserved and essential for glycan binding. In LLY the carboxy terminal lectin domain would be fused to the amino terminus of the CDC, orienting the binding site towards the upper surface of domain 1 of the CDC structure, potentially occluding the glycan-binding site. An assessment of the packing quality of the LLY^{CDC} structural model using ANOLEA (29) shows that residues in domain 1, near the predicted site of interaction between the amino terminal fucolectin domain and domain 1 of the CDC, exhibit a non-favorable energy environment. This suggests that these residues do not pack similarly to the analogous residues in ILY. Hence, the residues in this region may adopt a different conformation in LLY so that they can specifically interact with the glycan-binding surface of the lectin domain in the soluble monomer.

Our studies revealed that platelets from different donors responded differently to LLY-dependent pore-formation. Ohkuni et al. also observed a similar phenomenon in their aggregation experiments (7) (which we now know to be due to platelet lysis). The EC₅₀ for LLY on the platelets of a high responder donor was about six times lower than that observed when the platelets from a low responder when they were treated with LLY. This difference was abolished if both sets of platelets were treated with the lectin deficient LLY^{CDC}, or if high responder platelets were first treated with LLY^{Lec} or L-fucose before adding ILY. When the high responder platelets were pretreated with LLY^{Lec} to block its binding sites before the addition of LLY we observed more than a 10-fold increase in the EC₅₀. Hence, LLY exhibits an intrinsic pore-forming activity that is enhanced by the presence of the lectin domain, apparently in a glycan-dependent manner. The most likely explanation for the differences platelet sensitivity is that the levels of glycan recognized by the lectin domain differ in platelets from various donors. Based on the array analysis Le^{y} and/or Le^{b} antigens are candidates for these differences. The mechanistic stage of the LLY pore-forming mechanism that is modulated by the lectin domain is not yet known, but apparently occurs after the initial domain 4-mediated cell binding. Most toxins that have a lectin-like domains use it to bind to glycan-containing receptors (30-36), whereas the LLY lectin domain does not appear to participate in receptor recognition.

The glycan microarray analysis showed Le^{y} and Le^{b} are preferred ligands of the LLY lectin, with the H-antigen itself being a weaker ligand. The H-antigen is expressed in many cells and tissues of all individuals, but expression of Le^{y} and Le^{b} antigens is more restricted in adults. Besides being on erythrocytes and in body secretions (e.g. saliva), they are expressed throughout the gastrointestinal tract, but at levels that can differ based on location (37, 38). Expression of both antigens is often highly upregulated on epithelial tumors of the GI tract (39, 40). In normal tissue, Le^{b} is highly expressed on mucosal surfaces of the fetal colon, but in adults its expression is restricted to the proximal colon (41). Le^{y} is expressed strongly in proximal regions of the terminal ileum and cecum, and weakly in the ascending colon and beyond (42). In addition, both Le^{y} and Le^{b} can be found weakly expressed in gastric mucosa (43). Le^{y} is also expressed by some Helicobacter pylori isolates (44).

Interestingly, Le^{y} is expressed at low levels in circulating human peripheral blood granulocytes, but is upregulated upon their activation (45). Le^{y}-expressing neutrophils are also found in the synovial fluid of patients with arthritic joint disease (46). The mitis group of streptococci is part of the normal flora of the oropharynx and nasopharyx, but as indicated in the introduction they are increasingly associated with antibiotic-resistant bacteremia or septicemia. The lectin-dependent enhancement of LLY activity may increase its activity towards Le^{y} expressing activated neutrophils, thus potentially blunting one of the early responses to infection.

In summary, this work shows that LLY is a member of the CDC family of pore-forming toxins and is the only identified CDC that has a glycan-binding lectin domain. The lectin domain exhibits a strong preference for Le^{b} and Le^{y} and modulates LLY pore-forming activity in a glycan-dependent manner after LLY has bound to the membrane. Finally, several species of streptococci appear to carry the gene for LLY. One S. pneumoniae isolate appeared to carry the gene for LLY suggesting that there may be a subpopulation of this important pathogen that express LLY in association with PLY or it has been substituted for PLY.

### Utility

The proteins and protein fragments and variants thereof of the present invention have diagnostic and therapeutic uses.

In one aspect of the invention, the LLY-fucolectin domain (SEQ ID NO:3) of the present invention (or H-antigen binding fragments thereof) can be used for example in the identification and quantification of eukaryotic or prokaryotic glycans that contain Fucα1-2Galβ, particularly Lewis b (Le^{b}) and Lewis y (Le^{y}) antigens. Its fucolectin activity is unique, no other known lectin or monoclonal antibody exhibits its glycan specificity. These polypeptides can be used for any purpose where the specific identification and quantification of these glycans is required. One application is the quantification of these antigens on the surface of specific tumors types to help grade the tumor or identify the tumor (in vitro or in vivo). For example, Lewis y, one of the primary targets of the LLY-fucolectin domain, is also expressed preferentially on 70% of epithelial cell derived cancers such as, but not limited to, colon, breast, non-small cell lung, and ovary cancers. Thus, the LLY-fucolectin domain can be used as a diagnostic tool for identification and classification of a variety of such cancer types.

The LLY-fucolectin domain can also be used to target specific anti-tumor agents to tumors exhibiting expression or increased expression of these glycans (such as, but not limited to, those listed herein below). In particular, anticancer drugs may be coupled to the LLY-fucolectin domain and then targeted to tumors that express these glycans. Alternatively, these polypeptides could be used to decorate the surface of tumor cells (in vivo or in vitro) followed by the administration of an antibody raised against the LLY-fucolectin polypeptides. In vivo, this would then trigger the complement mediated lysis of the tumor cell. Another potential application is the use of these polypeptides to disrupt the interaction of H. pylori with its gastric receptors, Lewis y (Le^{y}) and Lewis b (Le^{b}).

In vitro the antibody could carry a reporter probe (e.g. fluorescent) for linking to the H-antigen binding fragment bound to the cell surface for identifying cells carrying the H-antigen (e.g., Le^{y} or Le^{b}). As shown herein and discussed in more detail below, we “engineered in” a cysteine for labeling with sulfhydryl-specific reagents (such as fluorescent probes) and shown that it can be efficiently labeled and used to detect these glycans.

Further, the present invention contemplates any protein sequence or fragment comprising amino acid substitutions of SEQ ID NO:3 which does not eliminate the binding affinity or specificity of the fucolectin for these antigens and uses thereof.

The present invention contemplates any nucleic acid which encodes SEQ ID NO:1 or SEQ ID NO:3 or which encodes any variant or alternate version of SEQ ID NO:3 or a fragment thereof as contemplated herein which functions as a Fucα1-2Galβ specific fucolectin particularly which binds with high specificity to Le^{y} and Le^{b}.

The present invention contemplates further any vector, host cell, plasmid, or expression system having a nucleic acid that encodes the LLY-fucolectin domain (or H-antigen binding fragment) of the present invention as contemplated or described herein.

The present invention also includes a purified nucleic acid segment encoding the protein component of the conjugate described herein above, a recombinant vector comprising such a purified nucleic acid segment, and a recombinant host cell comprising the recombinant vector.

The present invention further includes a pharmaceutical composition that comprises a pharmaceutically acceptable carrier, such as is described elsewhere herein, and a therapeutically effective or diagnostically-effective amount of the conjugate described herein above.

The present invention contemplates compositions of the novel polypeptides and variants described herein conjugated with a reporter moiety and uses thereof in a method of identification of cells or samples having Fucα1-2Galβ-containing glycans, specifically Lewis y and Lewis b antigens.

The present invention also contemplates any mutations in the glycan binding site of the LLY-fucolectin domain that may alter its binding specificity such that it would bind only Lewis y or Lewis b antigens or that alter its affinity for these substrates.

Where used herein the term “agent” refers to a conjugate of the invention.

Accordingly, the LLY-fucolectin polypeptides described herein are contemplated for use as a conjugate with an anticancer agent for treating a tumor which expresses Lewis y and Lewis b. In one aspect of the invention, a method for treating a subject having a condition characterized by an abnormal mammalian cell proliferation is provided. As used herein, subject means a mammal including humans, nonhuman primates, dogs, cats, sheep, goats, horses, cows, pigs and rodents and other livestock mammals or zoo animals. An abnormal mammalian cell proliferation disorder or condition, as used herein, refers to a localized region of cells (e.g., a tumor) which exhibit an abnormal (e.g., increased) rate of division as compared to their normal tissue counterparts.

As used herein, the term “conjugate” refers to a molecule that contains at least one receptor-binding ligand (LLY-fucolectin domain or binding fragment thereof) and at least one reporter moiety or anticancer agent that are coupled directly or via a linker. The conjugate may be produced by chemical coupling methods or by recombinant expression of chimeric DNA molecules to produce fusion proteins. The LLY-fucolectin domain may be all or a fragment of SEQ ID NO:3, or may be modified to enable linkage of the anticancer agent, for example by substitution with a linking amino acid (e.g., cysteine or others described herein) or with a linker or spacer such as is well known in the art for coupling moiety groups to proteins or peptides.

In one embodiment, the fucolectin polypeptide of the invention contemplated herein is an isolated polypeptide consisting of SEQ ID NO:3, or consisting of a fragment thereof, wherein the fragment comprises one of SEQ ID NO:16 through SEQ ID NO:25 (inclusive). In another embodiment, the invention is an isolated peptide consisting of a fragment of SEQ ID NO:1, wherein the fragment comprises one of SEQ ID NO:3 or at least one of SEQ ID NO:16 through SEQ ID NO:25 (inclusive). The invention further comprises a recombinant nucleic acid, consisting of a nucleic acid which encodes a fragment of SEQ ID NO:1, wherein the encoded fragment comprises SEQ ID NO:3 or a fragment thereof such as SEQ ID NO:16 through SEQ ID NO:25 (inclusive). The polypeptide may comprise substituted forms of SEQ ID NO:3 or fragments thereof or nucleic acids thereof which encode said polypeptides or fragments thereof.

In a preferred embodiment, the fucolectin of the invention comprises a purified polypeptide which is at least 90% identical to SEQ ID NO:3, or is at least, 91%, 92%, 93%, 94%, 95%, 96%, 97%, 98%, or 99% identical (in amino acid identity) to SEQ ID NO:3, and binds to Lewis y and Lewis b antigens. The purified polypeptide in another embodiment comprises a truncated fragment of SEQ ID NO:3 wherein 1, 2, 3, 4, 5, 6, 7, 8, 9, 10, 11 or 12 amino acid residues are truncated from the N-terminal end of SEQ ID NO:3 and/or 0, 1, 2, 3, 4, 5, 6, 7, 8, 9, 10, 11 or 12 amino acid residues are truncated from the C-terminal end of SEQ ID NO:3. Alternatively, the purified polypeptide may comprise a variant of SEQ ID NO:3 wherein one or more amino acids substitutions are made in the twelve N-terminal most amino acids and/or C-terminal most amino acids for inserting a linking amino acid such as cysteine (or other amino acid used for such reasons by these of ordinary skill in the art) for linking the reporter molecule or anticancer agent for forming the conjugate of the present invention.

The fucolectin of the present invention may comprise a fragment of SEQ ID NO:3, wherein the fragment may comprise amino acids 45-90 thereof (SEQ ID NO:16), amino acids 40-95 thereof (SEQ ID NO:17, amino acids 35-100 (SEQ ID NO:18), amino acids 30-110 (SEQ ID NO:19), amino acids 25-120 (SEQ ID NO:20), amino acids 20-130 (SEQ ID NO:21), amino acids 15-140 (SEQ ID NO:22), amino acids 10-150 (SEQ ID NO:23), amino acids 5-155 (SEQ ID NO:24), or amino acids 3-160 (SEQ ID NO:25).

As used herein, the term “covalently coupled”, “linked”, “bonded”, “joined”, and the like, with reference to the ligand and anticancer agent components of the conjugates of the present invention, mean that the specified components are either directly covalently bonded to one another or indirectly covalently bonded to one another through an intervening moiety or components, such as a bridge, spacer, linker or the like. For example, but not by way of limitation, the ligand and the anticancer agent may be chemically coupled together via a thioether linkage (49).

As used herein, the term “anticancer agent” refers to a molecule capable of inhibiting cancer cell function. The agent may inhibit proliferation or may be cytotoxic to cells. A variety of anticancer agents can be used and include those that inhibit protein synthesis and those that inhibit expression of certain genes essential for cellular growth or survival. Anticancer agents include those that result in cell death and those that inhibit cell growth, proliferation and/or differentiation. Preferably, the anticancer agent is selectively toxic against certain types of cancer cells but does not affect or is less effective against other normal cells. For example but not by way of limitation, the anticancer agent may be a protein which degrades a nonessential amino acid wherein the nonessential amino acid is still required for growth of tumor cells, such as but not limited to, methioninase and asparaginase. In another embodiment, the anticancer agent is an antineoplastic agent.

The conjugate of the present invention provides several advantages of the methodologies of the prior art. In particular since the anticancer agent is being targeted to cells that it is intended to kill, the dosages of the anticancer agent in the conjugate containing the anticancer agent should be significantly lower than when the anticancer agent alone is administered systemically.

The anticancer agent can be linked to any N-terminal amino acid or C-terminal amino acid of the fucolectin protein or peptide, and/or can be linked to an amino acid residue downstream of the N-terminal amino acid or C-terminal amino acid, such as lysine, histidine, tryptophan, aspartic acid, glutamic acid, serine, threonine, methionine, tyrosine, and cysteine, for example or other such linkable amino acids known to those of skill in the art. Cysteine-pegylated linking proteins or peptides, for example, are created by attaching a polyethylene glycol moeity to a thio group on a cysteine residue of the linking protein or peptide.

In other embodiments of the invention, the fucolectin protein, or peptide can be covalently linked to the anticancer agent via an intermediate linking moiety, or directly thereto by linking an amino group on the protein or peptide linker to a functional group on the anticancer agent or to a functional group on the intermediate linking moiety. For example, Table 3 shows several potential covalent linkages, and the activation and coupling compound which can be used to form the covalent linkage.

TABLE 3

| | Group Coupled | |
|---|---|---|
| Activation and Coupling | Intermediate Linker Moiety | Group on Protein |
| Method | Functional Group | or Peptide |
| Glutaraldehyde | Amide | Amino |
| Cyanogen bromide | Hydroxyl | Amino |
| Hydrazine | Amide | Amino |
| Benzoquinone | Hydroxyl | Amino |
| Periodate | Polysaccharide | Amino |
| Trichloro-s-triazine | Hydroxyl | Amino |
| Diazonium | Hydroxyl | Amino |
| Carbonyldiimidazole | Hydroxyl | Tyrosine |
| Tosylates | Hydroxyl | Amino |

Other methods for linking the protein or peptide linker of the present invention to the SWNT (or carbon nantoube) or the intermediate linking moiety include linkage to anhydride groups on the SWNT or intermediate linking moiety (e.g., see Srere et al. (51)). Alternatively, the linkage may be made to an acyl azide-activated material (51). The activation, for example, is performed first by esterification to yield the methyl ester; this is followed by hydrazinolysis to form the hydrazide. The hydrazide is allowed to react with nitrous acid to form the acyl azide. The acyl azide can then react with the nucleophilic groups, sulfhydryl, amino or hydroxyl, to yield the thioester, amide or ester linkage.

In another alternate method, linkage may occur via reaction of amino groups of the protein with the N-hydroxysuccinimide ester of PEG carboxylic acids. This is a common method for coupling PEG to proteins. In another method, 1-pyrenebutanoyl succinimide could be used as an intermediate linking moiety adsorbed to the SWNT then reacted with the protein or peptide linker. Further, PEGs with aldehyde groups could be linked to N-terminal amino groups on the protein or peptide linkers, or another intermediate linking moiety with aldehyde groups could be used. This method is particularly desirable since the linkage is primarily at the N-terminus of the protein or peptide.

Other methods can be used to link the fucolectin protein or peptide of the present invention directly to the anticancer agent, or indirectly thereto via an intermediate linking moiety. For example, proteins and peptides can be linked via their reactive residues which include the t-amino of L-lysine (L-Lys) and N-terminus amino group thiol of L-cysteine (L-Cys), carboxyl of L-aspartate (L-Asp) and L-glutamate (L-Glu) and C-terminus carboxyl group, phenolic of L-tyrosine (L-Tyr), guanidino of L-arginine (L-Arg), imidazole of L-histidine (L-His), disulfide of L-cystine, indole of L-tryptophan (L-Trp), thioether of L-methionine (L-Met), and hydroxyl of L-serine (L-Ser) and L-threonine (L-Thr).

Cellulose and cellulose derivatives which can be used as intermediate linking moieties in the present protein anticancer agent complexes include for example 4-aminobenzyl-cellulose, aminoethyl cellulose, diethylaminoethyl cellulose, epichlorohydrin triethanolamine-cellulose, oxy-cellulose, phospho-cellulose, sulfoethyl-cellulose, triethylaminoethyl-cellulose, triazinyl-cellulose, bromacetyl-cellulose, cellulose trans-2,3-carbonate, cellulose imidocarbonate, cellulose azide, cellulose carbonyl, diazo-cellulose, and isocyanat-cellulose.

In one embodiment, CMC, HEC, or HPC are treated for use as anchors for biological molecules by chemical conversion of all or some of the functional groups on the polymer, and are used to prepare stable suspensions. It is possible to convert the carboxylate functionalities of CMC to aldehydes using a variety of methods. For example the carboxylic acid of CMC can be converted to the acid chloride by thionyl chloride and then reduced to the aldehyde via the Rosenmund catalysts. HEC can be converted to the appropriate functional group by oxidizing a number of the terminal alkyl moieties using pyridinium dichromate in dichloremethane. Hydroxypropyl cellulose (HPC) can be utilized and functionalized in a manner identical to that of HEC.

Other coupling reactions which can be used herein to link the linking groups of proteins to functional groups thereon or on the anticancer agent or intermediate linking moieties include but are not limited to diazotization, amide (peptide) bond formation, alkylation and arylation, Schiff's base formation, Ugi reaction, amidination reactions, thiol-disulfide interchange reactions, mercury-enzyme interactions, and y-irradiation induced coupling.

Examples of the reactive groups on the anticancer agents or intermediate linking moieties which react in these coupling reactions include but are not limited to diazonium salt, acid anhydride, acyl azide, imidocarbonate, isothiocyanate, isocyanate, acyl chloride, cyclic carbonate, O-acylisourea, Woodward's reagent K derivative, δ-fluoramdinitroanilide, triazinyl, oxirane, vinylsulfonyl, vinyl keto, aldehyde, imine, imidoester, cyanide, disulfide residue, mercury derivative, matrix radical, amine, and acylhydrazide. Further explanation of these linking methods and linking groups can be found in (50).

Additionally the ligands of the present antibodies of the invention may be labelled with labels that produce a detectable signal (either in vitro or in vivo). Some labels, e.g. radionucleotides may produce a detectable signal and have a therapeutic property. Examples of other detectable labels include a fluorescent chromophore such as fluorescein, phycobiliprotein or tetraethyl rhodamine for fluorescence microscopy, an enzyme which produces a fluorescent or colored product for detection by fluorescence, absorbance, visible color or agglutination, which produces an electron dense product for demonstration by electron microscopy; or an electron dense molecule such as ferritin, peroxidase or gold beads for direct or indirect electron microscopic visualization.

The present invention also provides for a variety of methods for detecting cancer cells. These methods involve the administration to a patient. One method of detecting cancer cells in a patient involves the step of administering a labeled conjugate (labelled with a detectable label) to a human and subsequently detecting bound labeled conjugate by the presence of the label. Alternatively the conjugate may be administered to a sample from the patient for in vitro analysis thereof.

Conditions characterized by an abnormal mammalian cell proliferation, as the term is used herein, include but are not limited to conditions involving solid tumor masses of benign, pre-malignant or malignant character. Although not wishing to be bound by a particular theory or mechanism, some of these solid tumor masses may arise from at least one genetic mutation, some may display an increased rate of cellular proliferation as compared to the normal tissue counterpart, and still others may display factor independent cellular proliferation. Factor independent cellular proliferation is an example of a manifestation of loss of growth control signals which some, if not all, tumors or cancers undergo.

In one aspect, the invention provides a method for treating subjects having a condition characterized by an abnormal epithelial cell proliferation. Epithelial cells are cells occurring in one or more layers which cover the entire surface of the body and which line most of the hollow structures of the body, excluding the blood vessels, lymph vessels, and the heart interior which are lined with endothelium, and the chest and abdominal cavities which are lined with mesothelium. Examples of epithelium include anterius corneae, anterior epithelium of cornea, Barrett's epithelium, capsular epithelium, ciliated epithelium, columnar epithelium, epithelium corneae, corneal epithelium, cubical epithelium, cubical epithelium, cuboidal epithelium, epithelium eductus semicircularis, enamel epithelium, false epithelium, germinal epithelium, gingival epithelium, glandular epithelium, glomerular epithelium, laminated epithelium, epithelium of lens, epithelium lentis, mesenchymal epithelium, olfactory epithelium, pavement epithelium, pigmentary epithelium, pigmented epithelium, protective epithelium, pseudostratified epithelium, pyramidal epithelium, respiratory epithelium, rod epithelium, seminiferous epithelium, sense epithelium, sensory epithelium, simple epithelium, squamous epithelium, stratified epithelium, subcapsular epithelium, sulcular epithelium, tessellated epithelium, and transitional epithelium.

Another category of conditions characterized by abnormal epithelial cell proliferation is tumors of epithelial origin. Thus, in one aspect, the invention provides a method for treating subjects having epithelial tumors, especially these which overexpress Lewis y and Lewis b antigens or their surfaces. Epithelial tumors are known to those of ordinary skill in the art and include, but are not limited to, benign and premalignant epithelial tumors, such as breast fibroadenoma and colon adenoma, and malignant epithelial tumors. Malignant epithelial tumors include primary tumors, also referred to as carcinomas, and secondary tumors, also referred to as metastases of epithelial origin. Carcinomas intended for treatment with the methods of the present invention include, but are not limited to, acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epithelial adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparrum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypernephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the invention are used, for example, to treat subjects having cancer of the breast, cervix, ovary, prostate, lung, colon and rectum, pancreas, stomach or kidney.

The methods of the invention are also directed towards the treatment of subjects with metastatic tumors, preferably of epithelial origin. Carcinomas may metastasize to bone, as has been observed with breast cancer, and liver, as is sometimes the case with colon cancer. The methods of the invention are intended to treat metastatic tumors regardless of the site of the metastasis and/or the site of the primary tumor. In preferred embodiments, the metastases are of epithelial origin.

The compositions and methods of the invention in certain instances may be useful for replacing existing surgical procedures or drug therapies, although in many instances the present invention is useful in improving the efficacy of existing therapies for treating such conditions. Accordingly, combination therapy may be used to treat the subjects. For example, the agent may be administered to a subject in combination with another anticancer therapy. Suitable anticancer therapies include surgical procedures to remove the tumor mass, chemotherapy or localization radiation. The other anticancer therapy may be administered before, concurrent with, or after treatment with the therapeutic composition of the invention. There may also be a delay of several hours, days and in some instances weeks between the administration of the different treatments, such that the present agent may be administered before or after the other treatment.

As an example, the therapeutic conjugate of the present invention (above referred to herein as “agent”) may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. The agent may be administered either by continuous infusion or in a single bolus. Administration during or immediately after surgery may include a lavage, soak or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

In a preferred embodiment, the agent of the invention comprises an anticancer agent or drug conjugated to the entire LLY-fucolectin domain or to a functional fragment thereof as described elsewhere herein. Suitable anticancer drugs which may be used herein include but are not limited to: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycopherolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinrate Sulfate; Vinleurosine Sulfate; Vinorelbine Tartrate; Vinrosidine Sulfate; Vinzolidine Sulfate; Vorozole; Zeniplatin; Zinostatin; or Zorubicin Hydrochloride.

Other anticancer drugs which may be used herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; arnsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycdin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexametihylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxei analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazbloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzokane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromrelysin inhibitors; sulfinosine; superactive vasoactive intestinal peptide antagonist; suradista; suramin; swainsonine; synthetic glycosaminoglycans; tallimustine; tarnoxifen methiodide; tauromustine; tazarotene; tecogalan sodium; tegafur; tellurapyrylium; telomerase inhibitors; temoporfin; temozolomide; teniposide; tetrachlorodecaoxide; tetrazornine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Anticancer supplementary potentiating compounds may be used with the compositions described herein and include but are not limited to: Tricyclic anti-depressant drugs (e.g., imipramine, desipramine, amitryptyline, clornipramine, trimipramine, doxepin, nortriptyline, protriptyline, amoxapine and maprotiline); non-tricyclic anti-depressant drugs (e.g., sertraline, trazodone and citalopram); Ca⁺⁺. antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and multiple drug resistance reducing compounds such as Cremiaphor EL.

Other compounds which are useful in combination therapy for the purpose of the invention include the antiproliferation compound, Piritrexim Isethionate; the antiprostatic hypertrophy compound, Sitogluside; the benign prostatic hyperplasia therapy compound, Tamsulosin Hydrochloride; the prostate growth inhibitor, Pentomone; radioactive compounds such as Fibrinogen I 125, Fludeoxyglucose F 18, Fluorodopa F 18, Insulin I 125, Insulin I 131, Iobenguane I 123, Iodipamide Sodium I 131, Iodoantipyrine I 131, Iodocholesterol I 131, Iodohippurate Sodium I 123, Iodohippurate Sodium I 125, Iodohippurate Sodium I 131, Iodopyracet I 125, Iodopyracet I 131, Iofetamine Hydrochloride I 123, Iomethin I 125, Iomethin I 131, Iothalamate Sodium I 125, Iothalamate Sodium I 131, Iotyrosine I 131, Liothyronine I 125, Liothyronine I 131, Merisoprol Acetate Hg 197, Merisoprol Acetate Hg 203, Merisoprol Hg 197, Selenomethionine Se 75, Technetium Tc 99m Antimony Trisulfide Colloid, Technetium Tc 99m Bicisate, Technetium Tc 99m Disofenin, Technetium Tc 99m Etidronate, Technetium Tc 99m Exametazime, Technetium Tc 99m Furifosmin, Technetium Tc 99m Gluceptate, Technetium Tc 99m Lidofenin, Technetium Tc 99m Mebrofenin, Technetium Tc 99m Medronate, Technetium Tc 99m Medronate Disodium, Technetium Tc 99m Mertiatide, Technetium Tc 99m Oxidronate, Technetium Tc 99m Pentetate, Technetium Tc 99m Pentetate Calcium Trisodium, Technetium Tc 99m Sestamibi, Technetium Tc 99m Siboroxime, Technetium Tc 99m Succimer, Technetium Tc 99m Sulfur Colloid, Technetium Tc 99m Teboroxime, Technetium Tc 99m Tetrofosmin, Technetium Tc 99m Tiatide, Thyroxine I 125, Thyroxine I 131, Tolpovidone I 131, Triolein I 125, and Triolein I 131.

The agents of the invention are administered in therapeutically effective amounts. An effective amount is a dosage of the agent sufficient to provide a medically desirable result such as inhibition or reduction of tumor size or growth. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment. For example, an effective amount to inhibit cancer would be an amount sufficient to reduce or halt altogether the abnormal cell proliferation so as to slow or halt the development of or the progression of a cell mass such as, for example, a tumor. As used in the embodiments, “inhibit” embraces all of the foregoing.

Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount typically varies from 0.1 μg/kg to about 1000 mg/kg, preferably from about 0.01 mg/kg to about 500 mg/kg, and most preferably from about 0.1 mg/kg to about 100 mg/kg, in one or more dose administrations daily, for one or more days. In some embodiments, the agents are administered for more than 7 days, more than 10 days, more than 14 days and more than 20 days. In still other embodiments, the agent is administered over a period of weeks, or months. In still other embodiments, the agent is delivered on alternate days. For example, the agent is delivered every two days, or every three days, or every four days, or every five days, or every six days, or every week, or every month.

The agents of the invention can also be administered in prophylactically effective amounts, particularly in subjects diagnosed with benign or pre-malignant tumors. In these instances, the agents are administered in an amount effective to prevent the development of an abnormal mammalian cell proliferative mass or to prevent angiogenesis in the solid tumor mass, depending on the embodiment. The agents may also be administered in an amount effective to prevent metastasis of cells from a tumor to other tissues in the body. In these latter embodiments, the invention is directed to preventing the metastatic spread of a primary tumor.

According to another aspect of the invention, a kit is provided. The kit is a package which houses a container which contains an agent of the invention and also houses instructions for administering the agent of the invention to a subject having a condition characterized by an abnormal mammalian cell proliferation. The kit may optionally also contain one or more other anti-proliferative compounds or one or more anti-angiogenic compounds for use in combination therapies as described herein.

In still another aspect of the invention, kits for administration of an agent of the invention to a subject is provided. The kits include a container containing a composition which includes at least one agent of the invention, and instructions for administering the at least one agent to a subject having a condition characterized by an abnormal mammalian cell proliferation in an amount effective to inhibit proliferation. In certain embodiments, the container is a container for intravenous administration. In other embodiments the agent is provided in an inhaler. In still other embodiments, the agent is provided in a polymeric matrix or in the form of a liposome. In yet other embodiments, kits are provided for the administration of an agent of the invention to a subject having an abnormal mammalian cell mass for the purpose of inhibiting angiogenesis in the cell mass. In these latter kits, the agent is provided in an amount effective to inhibit angiogenesis along with instructions for use in subjects in need of such treatment. The agent may be provided in the kit in any form described elsewhere herein.

The agent may be administered alone or in combination with the above-described drug therapies by a variety of administration routes available. The particular mode selected will depend, of course, upon the agent selected, the condition being treated, the severity of the condition, whether the treatment is therapeutic or prophylactic, and the dosage required for efficacy. The methods of the invention, generally speaking, may be practiced using any mode of administration that is medically acceptable, meaning any mode that produces effective levels of the active compounds without causing clinically unacceptable adverse effects. The administration may, for example, be oral, intraperitoneal, intra-cavity such as rectal or vaginal, transdermal, topical, nasal, inhalation, mucosal, interdermal, or parenteral routes. The term “parenteral” includes subcutaneous, intravenous, intramuscular, or infusion. Intravenous or intramuscular routes may not particularly suitable for long term therapy and prophylaxis. In certain embodiments, however, it may be appropriate to administer the agent in a continuous infusion every several days, or once a week, or every several weeks, or once a month. Intravenous or intramuscular routes may be preferred in emergency situations. Oral administration may be used for prophylactic treatment because of the convenience to the patient as well as the dosing schedule. Likewise, sustained release devices as described herein may be useful in certain embodiments for prophylactic or post surgery treatment, for example.

When using the agent of the invention in subjects in whom the primary site of abnormal proliferation is well delineated and easily accessible, direct administration to the site may be preferred, provided the tumor has not already metastasized. For example, administration by inhalation for lung tumors or by suppositories in the treatment of cervical, ovarian or rectal tumors may be preferred. In still other embodiments aimed at the treatment of subjects with breast or prostate cancer, the agents may be delivered by injection directly into the tissue with, for example, a biopsy needle and syringe.

Systemic administration may be preferred in some instances such as, for example, if the subject is known to have or is suspected of having metastases. In this way, all tumor sites, whether primary or secondary may receive the agent. Systemic delivery may be accomplished through for example, oral or parenteral administration. Inhalation may be used in either systemic or local delivery, as described below.

As discussed earlier, the agent may also be delivered to a tumor site during or immediately after a surgical procedure to remove the tumor by lavage into the excision site or by perfusion of the affected tissue with a physiologically acceptable solution containing the agent. Alternatively, the patient may be administered the agent prior to or following the surgical procedure by continuous infusion. In yet other embodiments, a sustained release device, as described below, such as a polymeric implant may be positioned during surgery in the vicinity of the excision site so as to provide a high local concentration of the agent. These latter embodiments may be appropriate to prevent regrowth of the tumor.

The agent of the invention may be administered alone or in combination with the above-described drug therapies as part of a pharmaceutical composition. Such a pharmaceutical composition may include the agent in combination with any standard physiologically and/or pharmaceutically acceptable carriers which are known in the art. The compositions should be sterile and contain either a therapeutically or prophylactically effective amount of the agent in a unit of weight or volume suitable for administration to a subject. The term “pharmaceutically-acceptable carrier” as used herein means one or more compatible solid or liquid filler, vehicle, diluents or encapsulating substances which are suitable for administration into a subject of the invention. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically-acceptable further means a non-toxic material that is compatible with a biological system such as a cell, cell culture, tissue, or organism. The characteristics of the carrier will depend on the route of administration. Physiologically and pharmaceutically-acceptable carriers include diluents, vehicles, fillers, salts, buffers, stabilizers, solubilizers, and other materials which are well known in the art.

Compositions suitable for parenteral administration conveniently comprise a sterile aqueous preparation of the agent, which is preferably isotonic with the blood of the recipient. This aqueous preparation may be formulated according to known methods using suitable dispersing or wetting compounds and suspending compounds. The sterile injectable preparation also may be a sterile injectable solution or suspension in a non-toxic parenterally-acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulations suitable for oral, subcutaneous, intravenous, intramuscular, or other methods of administration can be found in Remington's Pharmaceutical Sciences, Mack Publishing Co., Easton, Pa.

Preparations for parenteral administration preferably include sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating compounds, and inert gases and the like. The pharmaceutical compositions may conveniently be presented in unit dosage form and may be prepared by any of the methods well-known in the art of pharmacy.

Compositions suitable for oral administration may be presented as discrete units, such as capsules, tablets, lozenges, each containing a predetermined amount of the agent. Other compositions include suspensions in aqueous liquids or non-aqueous liquids such as a syrup, elixir or an emulsion.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular or pulmonary surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polymethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, copolymers and mixtures thereof.

Bioadhesive polymers of particular interest include bioerodible hydrogels, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methactylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include timed release, delayed release or sustained release delivery systems. Such systems can avoid repeated administrations of the agent of the invention, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyesteramides, polyorthoesters, polyhydroxybutyric acid, and polyanhydrides. Microcapsules of the foregoing polymers may be used. Delivery systems may also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like.

Use of a long-term sustained release implant may be particularly suitable for treatment of chronic conditions, such as a the suspected presence of dormant metastases. Long-term release, as used herein, means that the implant is constructed and arranged to delivery therapeutic levels of the active ingredient for at least 30 days, at least 60 days and more preferably for several months. Long-term sustained release implants are well-known to those of ordinary skill in the art and include some of the release systems described above.

In the present invention, the agent is targeted to a site of abnormal cell proliferation, such as, an epithelical tumor, through the use of the presently described fucolectin or an effective portion thereof specific for tumor type which overexpress H-antigens. The fucolectin may be directly conjugated to the anticancer agents of the invention via a covalent linkage. The agent may be indirectly conjugated via a linker. Alternatively, the targeting compound may be conjugated or associated with an intermediary compound such as, for example, a liposome within which the agent is encapsulated.

The term “pharmaceutically acceptable esters” where used herein refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted-into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters.

The term “pharmaceutically acceptable salts” is art recognized and includes relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The language “pharmaceutical composition” includes preparations suitable for administration to mammals described herein, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient (fucolectin-anticancer agent conjugate) in combination with a pharmaceutically acceptable carrier.

Pharmaceutical compositions comprising compounds of the invention may contain wetting agents, emulsifiers and lubricants, such as sodium lauryl sulfate magnesium stearate, as well as coloring agents, release agents, coating agents, sweetening, flavoring and perfuming agents, and preservatives.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of agent which can be combined with a carrier material to produce a single dosage form will generally be that amount of the agent which produces a therapeutic effect. Generally, out of one hundred percent, this amount will range from about 1 percent to about ninety-nine percent of agent, preferably from about 5 percent to about 70 percent, most preferably from about 10 percent to about 30 percent.

Methods of preparing these formulations/or compositions include the step of bringing into association the agent of the present invention (fucolectin-anticancer agent conjugate) with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the agent of the present invention as an active ingredient. A compound of the present invention may also be administered as a bolus, electuary or paste.

In solid dosage forms of the invention for oral administration (capsules, tablets, pills, dragees, powders, granules and the like), the agent is mixed with one or more pharmaceutically acceptable carriers, such as, sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as capsules, pills and granules, may optionally be scored or prepared with coatings and shells, such as enteric coatings and other coatings well known in the pharmaceutical-formulating art. They may also be formulated, as noted above, so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention as described elsewhere herein include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain buffers, bacteriostats, solutes which render the formulation isotonic with the blood of the intended recipient or suspending or thickening agents.

Examples of suitable aqueous and nonaqueous carriers which may be employed in the pharmaceutical compositions of the invention as noted elsewhere herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

As described elsewhere herein, the preparations of the present invention may be given orally, parenterally, topically, rectally, intralesionally, intraorbitally, intracapsularly, directly instilled into a cavity, or by inhalation. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

These compounds may be administered to humans and other mammals for therapy by any suitable route of administrations including orally, nasally, as by, for example, a spray, rectally, intravaginally, parenterally; intracisternally and topically, as by powders, ointments or drops, including buccally and sublingually. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

The selected dosage level will depend upon a variety of factors including the activity of the particular compound of the present invention employed, or the ester, salt or amide thereof, the route of administration, the time of administration, the rate of excretion of the particular compound being employed; the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. While it is possible for a compound of the present invention to be administered alone, it is preferable to administer the compound as a pharmaceutical composition.

In one embodiment of the present invention, a substantially inert macromolecular intermediate linking moiety can be used to link the LLY-fucolectin domain protein to the anticancer agent for example by linking a functional group on the intermediate linking moiety to an amino group or side group of the fucolectin doman and to a linking group of the anticancer agent.

Since the LLY-fucolectin domains described herein are bacterially-derived proteins, the fucolectin of the conjugate of the present invention may be modified so as to reduce the immunogenicity thereof. One method for reducing a protein's immunogenicity and increasing serum half-life is to conjugate the protein to polyethylene glycol (PEG).

The term “polyethylene glycol” or “PEG” is also intended to include any other polyalkylene glycol compound or a derivative thereof, with or without coupling agents or derivatization with coupling or activating moeities (e.g., with thiol, triflate, tresylate, azirdine, oxirane, or preferably with a maleimide moiety). Compounds such as maleimido monomethoxy PEG are exemplary or activated PEG compounds of the invention. Other polyalkylene glycol compounds, such as polypropylene glycol, may be used in the present invention. Other appropriate polymer conjugates include, but are not limited to, non-polypeptide polymers, charged or neutral polymers of the following types: dextran, colominic acids or other carbohydrate based polymers, biotin deriviatives and dendrimers, for example. The term PEG is also meant to include other polymers of the class polyalkylene oxides.

The PEG can be linked to any N-terminal amino acid of the conjugate, and/or can be linked to an amino acid residue downstream of the N-terminal amino acid, such as lysine, histidine, tryptophan, aspartic acid, glutamic acid, and cysteine, for example or other such linkable amino acids known to those of skill in the art. Cysteine-pegylated conjugates, for example, are created by attaching polyethylene glycol to a thio group on a cysteine residue of the conjugate.

The PEG moiety attached to the conjugate may range in molecular weight, (though is not limited to) for example, from about 200 to 20,000 MW.

The conjugates contemplated herein can be adsorbed or linked to PEG molecules using techniques shown, for example (but not limited to), in U.S. Pat. Nos. 4,179,337; 5,382,657; 5,972,885; 6,177,087; 6,165,509; 5,766,897; and 6,217,869; and Published Application 200610275371; the specifications and drawings each of which are hereby expressly incorporated by reference herein in its entirety.

Another method for reducing a protein's immunogenicity is liposome encapsulation using methods known in the art.

These techniques will greatly reduce the immunological response to the conjugate without significant effect on effectiveness of the conjugate. Liposome encapsulation has the advantage that covalent attachment of moieties to the enzyme is not required, which may be helpful to preserve binding of the proposed conjugates to the receptors on cancer cells.

While the invention has been described herein in connection with certain preferred embodiments in the examples herein so that aspects thereof may be more fully understood and appreciated, it is not intended to limit the invention to these particular embodiments. On the contrary, it is intended to cover all alternatives, modifications and equivalents as may be included within the scope of the invention as defined herein. Thus, these examples, which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention. For example, although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process and compositions, methods and steps described in the specification. As one of ordinary skill in the art will readily appreciate from the disclosure of the present invention, processes, compositions, methods, or steps, presently existing or later to be developed that perform substantially the same function or achieve substantially the same result as the corresponding embodiments described herein may be utilized according to the present invention. Accordingly, the appended claims are intended to include within their scope such processes, compositions, methods, or steps.

Abbreviations used herein:
LLY: lectinolysin, ILY: intermedilysin; PLY: pneumolysin, PFO: perfringolysin, SM-hPAF: Streptococcus mitis human platelet aggregation factor; EC₅₀: half maximal effective concentration; LLY^{CDC}: cholesterol dependent cytolysin domain of LLY; LLY^{LEC}: fucolectin domain of LLY.

All patents, patent applications, references and other documents identified herein are incorporated in their entirety herein by reference.

### REFERENCES

1. Kennedy, M. J., Jackson, M. A., and Kearns, G. L. (2004) Delayed diagnosis of penicillin-resistant Streptococcus mitis endocarditis following single-dose amoxicillin prophylaxis in a child, Clin Pediatr (Phila) 43, 773-776.
2. Gowda, R. M., Ansari, A. W., and Khan, I. A. (2003) Complete endocardial cushion defect (complete atrioventricular canal) manifested in adult life by Streptococcus mitis endocarditis of the common atrioventricular valve, Int J Cardiology 89, 109-110.
3. Hall, G. E., and Baddour, L. M. (2002) Apparent failure of endocarditis prophylaxis caused by penicillin-resistant Streptococcus mitis, Amer J Med Sci 324, 51-53.
4. Huang, I. F., Chiou, C. C., Liu, Y. C., and Hsieh, K. S. (2002) Endocarditis caused by penicillin-resistant Streptococcus mitis in a 12-year-old boy, J Microbiol, Immunol, Infect (China) 35, 129-132.
5. Lu, H. Z., Weng, X. H., Zhu, B., Li, H., Yin, Y. K., Zhang, Y. X., Haas, D. W., and Tang, Y. W. (2003) Major outbreak of toxic shock-like syndrome caused by Streptococcus mitis, J Clin Microbiol 41, 3051-3055.
6. Jefferies, J., Nieminen, L., Kirkham, L. A., Johnston, C., Smith, A., and Mitchell, T. J. (2007) Identification of a Secreted Cholesterol-Dependent Cytolysin (Mitilysin) from Streptococcus mitis, J Bacteriol 189, 627-632.
7. Ohkuni, H., Todome, Y., Okibayashi, F., Watanabe, Y., Ohtani, N., Ishikawa, T., Asano, G., and Kotani, S. (1997) Purification and partial characterization of a novel human platelet aggregation factor in the extracellular products of Streptococcus mitis, strain Nm-65, FEMS Immunol Med Microbiol 17, 121-129.
8. Ohkuni, H., Todome, Y., Takahashi, H., Nagamune, H., Abe, J., Ohtsuka, H., and Hatakeyama, H. (2006) Antibody titers to Streptococcus mitis-derived human platelet aggregation factor (Sm-hPAF) in the sera of patients with Kawasaki disease, in Proceedings of the XVIth Lancefield International Symposium on Streptococci and Streptococcal Diseases (Sriprakash, K. S., Ed.), pp 71-74, Elsevier B.V., Palm Cove, Aus.
9. Nagamune, H., Whiley, R. A., Goto, T., Inai, Y., Maeda, T., Hardie, J. M., and Kourai, H. (2000) Distribution of the intermedilysin gene among the anginosus group streptococci and correlation between intermedilysin production and deep-seated infection with Streptococcus intermedius, J Clin Microbiol 38, 220-226.
10. Bianchet, M. A., Odom, E. W., Vasta, G. R., and Amzel, L. M. (2002) A novel fucose recognition fold involved in innate immunity, Nat Struct Biol 9, 628-634.
11. Majumder, S., Roy, A., and Mandal, C. (2004) Prediction of 3-D structures of fucose-binding proteins and structural analysis of their interaction with ligands, Glycoconj J 20, 545-550.
12. Giddings, K. S., Johnson, A. E., and Tweten, R. K. (2003) Redefining cholesterol's role in the mechanism of the cholesterol-dependent cytolysins, Proc Natl Acad Sci USA 100, 11315-11320.
13. Shepard, L. A., Heuck, A. P., Hamman, B. D., Rossjohn, J., Parker, M. W., Ryan, K. R., Johnson, A. E., and Tweten, R. K. (1998) Identification of a membrane-spanning domain of the thiol-activated pore-forming toxin Clostridium perfringens perfringolysin O: an a-helical to b-sheet transition identified by fluorescence spectroscopy, Biochemistry 37, 14563-14574.
14. Soltani, C. E., Hotze, E. M., Johnson, A. E., and Tweten, R. K. (2007) Specific protein-membrane contacts are required for prepore and pore assembly by a cholesterol-dependent cytolysin, J Biol Chem 282, 15709-15716.
15. Song, X., Xia, B., Lasanajak, Y., Smith, D. F., and Cummings, R. D. (2008) Quantifiable fluorescent glycan microarrays, Glycoconj J 25, 15-25.
16. Alberio, L., Safa, O., Clemetson, K. J., Esmon, C. T., and Dale, G. L. (2000) Surface expression and functional characterization of alpha-granule factor V in human platelets: effects of ionophore A23187, thrombin, collagen, and convulxin, Blood 95, 1694-1702.
17. Remenyi, G., Szasz, R., Friese, P., and Dale, G. L. (2005) Role of mitochondrial permeability transition pore in coated-platelet formation, Arterioscl, Thromb Vascul Biol 25, 467-471.
18. Shepard, L. A., Shatursky, O., Johnson, A. E., and Tweten, R. K. (2000) The mechanism of assembly and insertion of the membrane complex of the cholesterol-dependent cytolysin perfringolysin O: Formation of a large prepore complex, Biochemistry 39, 10284-10293.
19. Boraston, A. B., Wang, D., and Burke, R. D. (2006) Blood group antigen recognition by a Streptococcus pneumoniae virulence factor, J Biol Chem 281, 35263-35271.
20. Czajkowsky, D. M., Hotze, E. M., Shao, Z., and Tweten, R. K. (2004) Vertical collapse of a cytolysin prepore moves its transmembrane β-hairpins to the membrane, EMBO J 23, 3206-3215.
21. Harris, R. W., Sims, P. J., and Tweten, R. K. (1991) Kinetic aspects of the aggregation of Clostridium perfringens theta toxin on erythrocyte membranes: A fluorescence energy transfer study, J. Biol. Chem. 266, 6936-6941.
22. Hotze, E. M., Wilson-Kubalek, E. M., Rossjohn, J., Parker, M. W., Johnson, A. E., and Tweten, R. K. (2001) Arresting pore formation of a cholesterol-dependent cytolysin by disulfide trapping synchronizes the insertion of the transmembrane beta-sheet from a prepore intermediate, J Biol Chem 276, 8261-8268.
23. Giddings, K. S., Zhao, J., Sims, P. J., and Tweten, R. K. (2004) Human CD59 is a receptor for the cholesterol-dependent cytolysin intermedilysin, Nat Struct Mol Biol 12, 1173-1178.
24. Heuck, A. P., Tweten, R. K., and Johnson, A. E. (2003) Assembly and topography of the prepore complex in cholesterol-dependent cytolysins, J Biol Chem 278, 31218-31225.
25. Ramachandran, R., Heuck, A. P., Tweten, R. K., and Johnson, A. E. (2002) Structural insights into the membrane-anchoring mechanism of a cholesterol-dependent cytolysin, Nat Struct Biol 9, 823-827.
26. Ramachandran, R., Tweten, R. K., and Johnson, A. E. (2005) The domains of a cholesterol-dependent cytolysin undergo a major FRET-detected rearrangement during pore formation, Proc Natl Acad Sci 102, 7139-7144.
27. Tweten, R. K. (2005) The cholesterol-dependent cytolysins; a family of versatile pore-forming toxins, Infect Immun 73, 6199-6209.
28. Kawamura, Y., Hou, X. G., Sultana, F., Miura, H., and Ezaki, T. (1995) Determination of 16S rRNA sequences of Streptococcus mitis and Streptococcus gordonii and phylogenetic relationships among members of the genus Streptococcus, Int J Syst Bacteriol 45, 406-408.
29. Melo, F., and Feytmans, E. (1998) Assessing protein structures with a non-local atomic interaction energy, J Mol Biol 277, 1141-1152.
30. Merritt, E. A., Sixma, T. K., Kalk, K. H., van Zanten, B. A., and Hol, W. G. (1994) Galactose-binding site in Escherichia coli heat-labile enterotoxin (LT) and cholera toxin (CT), Mol Microbiol 13, 745-753.
31. Stein, P. E., Boodhoo, A., Armstrong, G. D., Cockle, S. A., Klein, M. H., and Read, R. J. (1994) The crystal structure of pertussis toxin, Structure 2, 45-57.
32. Olson, R., and Gouaux, E. (2005) Crystal structure of the Vibrio cholerae cytolysin (VCC) pro-toxin and its assembly into a heptameric transmembrane pore, J Mol Biol 350, 997-1016.
33. Clark, G. F., Krivan, H. C., Wilkins, T. D., and Smith, D. F. (1987) Toxin A from Clostridium difficile binds to rabbit erythrocyte glycolipids with terminal Gal alpha 1-3Gal beta 1-4GlcNAc sequences, Arch Biochem Biophys 257, 217-229.
34. Nesic, D., Hsu, Y., and Stebbins, C. E. (2004) Assembly and function of a bacterial genotoxin, Nature 429, 429-433.
35. Tateno, H., and Goldstein, I. J. (2003) Molecular cloning, expression, and characterization of novel hemolytic lectins from the mushroom Laetiporus sulphurous, which show homology to bacterial toxins, J Biol Chem 278, 40455-40463.
36. Rossjohn, J., Buckley, J. T., Hazes, B., Murzin, A. G., Read, R. J., and Parker, M. W. (1997) Aerolysin and pertussis toxin share a common receptor-binding domain, Embo J 16, 3426-3434.
37. Hakomori, S. I., and Siddiqui, B. (1974) Isolation and characterization of glycosphingolipid from animal cells and their membranes, Methods Enzymol 32, 345-367.
38. Marcus, D. M. (1969) The ABO and Lewis blood-group system. Immunochemistry, genetics and relation to human disease, N Engl J Med 280, 994-1006.
39. Inoue, M., Nakayama, M., and Tanizawa, O. (1990) Altered expression of Lewis blood group and related antigens in fetal, normal adult and malignant tissues of the uterine endometrium, Virchows Archie 416, 221-228.
40. Heller, D. S., and Thung, S. N. (1990) Expression of Lewis(x) and Lewis(y) blood group related antigens in fetal livers, Pediat Pathol 10, 681-687.
41. Jass, J. R., and Roberton, A. M. (1994) Colorectal mucin histochemistry in health and disease: a critical review, Pathol Internat 44, 487-504.
42. Abe, K., Hakomori, S., and Ohshiba, S. (1986) Differential expression of difucosyl type 2 chain (LeY) defined by monoclonal antibody AH6 in different locations of colonic epithelia, various histological types of colonic polyps, and adenocarcinomas, Cancer Res 46, 2639-2644.
43. Sakamoto, S., Watanabe, T., Tokumaru, T., Takagi, H., Nakazato, H., and Lloyd, K. O. (1989) Expression of Lewisa, Lewisb, Lewisx, Lewisy, siayl-Lewisa, and sialyl-Lewisx blood group antigens in human gastric carcinoma and in normal gastric tissue, Cancer Res 49, 745-752.
44. Appelmelk, B. J., Simoons-Smit, I., Negrini, R., Moran, A. P., Aspinall, G. O., Forte, J. G., De Vries, T., Quan, H., Verboom, T., Maaskant, J. J., Ghiara, P., Kuipers, E. J., Bloemena, E., Tadema, T. M., Townsend, R. R., Tyagarajan, K., Crothers, J. M., Jr., Monteiro, M. A., Savio, A., and De Graaff, J. (1996) Potential role of molecular mimicry between Helicobacter pylori lipopolysaccharide and host Lewis blood group antigens in autoimmunity, Infect Immun 64, 2031-2040.
45. Dettke, M., Palfi, G., and Loibner, H. (2000) Activation-dependent expression of the blood group-related lewis Y antigen on peripheral blood granulocytes, J Leukoc Biol 68, 511-514.
46. Dettke, M., Palfi, G., Pursch, E., Fischer, M. B., and Loibner, H. (2001) Increased expression of the blood group-related Lewis Y antigen on synovial fluid granulocytes of patients with arthritic joint diseases, Rheumatology (Oxford, England) 40, 1033-1037.
47. Polekhina, G., Giddings, K. S., Tweten, R. K., and Parker, M. W. (2005) Insights into the action of the superfamily of cholesterol-dependent cytolysins from studies of intermedilysin, Proc Natl Acad Sci 102, 600-605.
48. Humphrey, W., Dalke, A., and Schulten, K. (1996) VMD: visual molecular dynamics, J Mol Graph 14, 33-38, 27-38.
49. Mickisch, G. H., Pai, L. H., Siegsmund, M., Campain, J., Gottesman, M. M. and Pastan, I. (1993) Pseudomonas exotoxin conjugated to monoclonal antibody MRK16 specifically kills multidrug resistant cells in cultured renal carcinomas and in MDR-transgenic mice. J. Urology 149: 174-178.
50. J. M. S. Carbal and J. F. Kenndy “Covalent and Coordination Immobilization of Proteins” in “Protein Immobilization Fundamentals and Applications”, ed. R. F. Taylor, (1991), pp. 73-138, Marcel Dekker, Inc.
51. Srere, P. A. and Uyeda, K., Functional groups on enzymes suitable for binding to matrices, Methods in Enzymology. Volume XLIV. Immobilized Enzymes, K. Mosbach (ed.), Academic Press, NY, 1976.
52. Risler et al. “Amino Acid Substitutions in Structurally Related Proteins. A Pattern Recognition Approach.” J. Mol. Biol. 204:1019-1029 (1988).
53. Niefind et al. “Amino Acid Similarity Coefficients for Protein Modeling and Sequence Alignment Derived from Main-Chain Folding Anoles.” J. Mol. Biol. 219:481-497 (1991).
54. Overington et al. “Environment-Specific Amino Acid Substitution Tables: Tertiary Templates and Prediction of Protein Folds,” Protein Science 1:216-226 (1992).

What is claimed is:

1. A purified polypeptide consisting of SEQ ID NO:3, or consisting of a fragment thereof, wherein the fragment comprises SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25 or consisting of a polypeptide having a sequence which is at least 90% identical to SEQ ID NO:3 and binds to Lewis y and Lewis b antigens.

2. A purified polypeptide consisting of a fragment of SEQ ID NO:1, wherein the fragment comprises at least one of SEQ ID NO:3, SEQ ID NO:16, SEQ ID NO:17, SEQ ID NO:18, SEQ ID NO:19, SEQ ID NO: 20, SEQ ID NO:21, SEQ ID NO:22, SEQ ID NO:23, SEQ ID NO:24, or SEQ ID NO:25.

3. A purified polypeptide consisting of a fucolectin binding fragment of SEQ ID NO:1 wherein the fucolectin binding fragment comprises SEQ ID NO:16 or comprises a sequence at least 90% identical to amino acids 37-198 of SEQ ID NO:1, and binds to Lewis y and Lewis b antigens.

4. The purified polypeptide of claim 3 wherein the isolated fucolectin binding fragment comprises amino acids 37-198 of SEQ ID NO:1.

5. The purified polypeptide of claim 3 wherein the fucolectin binding fragment comprises a sequence which is at least 95% identical to amino acids 37-198 of SEQ ID NO:1.

6. A nucleic acid encoding the polypeptide of claim 1.

7. A vector comprising the nucleic acid of claim 6.

8. A host cell comprising the vector of claim 7.

9. A conjugate for treating or diagnosing an epithelial derived tumor or cancer cell, comprising:
the purified polypeptide of claim 1; and
an anticancer agent or reporter molecule operatively attached to the polypeptide.

10. The conjugate of claim 9 further comprising a linker for operatively attaching the anticancer agent or reporter molecule to the polypeptide.

11. A composition comprising the polypeptide of claim 1 disposed within a pharmaceutically acceptable carrier.

12. A composition comprising the conjugate of claim 9 disposed within a pharmaceutically-acceptable carrier.

13. A method of treating a subject having an epithelial derived cancerous condition, consisting:
providing a therapeutic conjugate comprising the purified polypeptide of claim 1 having an anticancer agent operatively attached thereto; and
administering to the subject a quantity of the therapeutic conjugate.
